(19) United States
(12) Patent Application Publication
(10) Pub. No.: US 2015/0051595 A1
(43) Pub. Date: Feb. 19, 2015

(54) DEVICES AND METHODS FOR DENERVATION OF THE NERVES SURROUNDING THE PULMONARY VEINS FOR TREATMENT OF ATRIAL FIBRILLATION

(71) Applicant: James Margolis, Coral Gables, FL (US)

(72) Inventor: James Margolis, Coral Gables, FL (US)

(21) Appl. No.: 14/457,943

(22) Filed: Aug. 12, 2014

Related U.S. Application Data
(60) Provisional application No. 61/867,237, filed on Aug. 19, 2013.

(51) Int. Cl. A61B 18/00; A61B 18/02; A61B 17/32; A61B 18/14; A61B 18/12

(52) U.S. Cl. CPC A61B 18/00; A61B 18/1492; A61B 18/1206; A61B 17/320068; A61B 18/02; A61B 2018/00791; A61B 2018/00827; A61B 2018/00892; A61B 2018/00875; A61B 2018/00648; A61B 2018/00702; A61B 2018/00351; A61B 2018/00434; A61B 2018/00601; A61B 2018/0022; A61B 2018/126; A61B 2018/1253; A61B 2017/320072; A61B 2018/00267; A61B 2018/0212
USPC 606/21; 606/39

(57) ABSTRACT

Methods, systems, and devices for providing a denervating energy treatment to the tissue of the pulmonary vein and antrum region of the left atrium utilizing a catheter-based structure having one or more energy delivery surfaces. In some instances energy delivery surfaces are arranged with a circumferential and axial offset relative to one another. A pattern of individual lesions loosely approximating a helix, or other staggered pattern, or roughly circumferential are placed so as to provide a pattern which covers substantially the circumference of the treated area while avoiding stenosis. Denervating energy is applied by modulation of the energy delivery surfaces using an energy source integrated with a controller and control algorithm. In some instances feedback is used in a control algorithm for energy modulation. Energy sources are radiofrequency, ultrasound, and cryogenic.

### CROSS REFERENCE TO RELATED APPLICATIONS

This application claims the benefit of U.S. Provisional Application 61/867,237 Devices and Methods for Denervation of the Nerves Surrounding the Pulmonary Veins for Treatment of Atrial Fibrillation, filed Aug. 19, 2013.

### BACKGROUND

Atrial fibrillation (“AF”) is the most common cardiac arrhythmia causing the muscles of the atria to contract in an irregular quivering motion rather than in the coordinated contraction that occurs during normal cardiac rhythm. AF may be detected by the presence of an irregular pulse or by the absence of p-waves on an electrocardiogram. During an episode of AF, the regular electrical impulses that are normally generated by the sinoatrial (SA) node are overwhelmed by rapid disorganized electrical impulses in the atria. These disorganized impulses are induced by “triggers” that are usually, though not always, located in and around the orifices of the pulmonary veins. Because the resultant disorganized impulses of AF reach the atrioventricular (AV) node in a rapid (up to 600 per minute) and highly irregular manner, the impulses that are subsequently filtered and conducted through the AV node to the ventricles are also rapid (around 150 per minute). AF episodes may be intermittent (“paroxysmal”) lasting from seconds to weeks or they may last for years, in which case the AF may be referred to as “chronic AF.” Untreated paroxysmal AF usually leads to chronic AF.

Although patients rarely experience immediate life-threatening problems from the onset of AF, they commonly experience immediate symptoms such as palpitations of the heart, weakness, tiredness, and shortness of breath. The most serious complication of AF is the risk of stroke caused by the pooling and stasis of blood in the left atrial appendage (LAA) that results in the formation of clots that may break off and travel to the brain. Patients with chronic AF lose up to 20% of their pumping capacity. This leads to chronic fatigue and even heart failure.

Paroxysmal atrial fibrillation may be treated by ablation of nerve fibers surrounding the pulmonary veins. Ablation of these nerve fibers has been demonstrated to prevent AF triggering events, and in many cases may cure the problem. In their present iterations, the procedures used to produce ablation patterns that isolate and prevent AF triggering events are lengthy invasive procedures that require a high degree of specialized operator skill, and which are frequently ineffective and not always permanent.

A myriad of devices using various forms of energy (RF devices, ultrasound and cryothermia) have been tried to simplify the procedure, to increase the completeness of ablation, and consequently the success rate. Confounding all of the devices is the trade-off between completeness of denervation and procedural complications—phrenic nerve palsy, esophageal rupture and pulmonary vein stenosis. The right phrenic nerve courses close to the right superior pulmonary vein (J Cardiovasc Electrophysiol. 2005 March; 16(3):309-13; the contents of which being incorporated herein by reference in their entirety), and the esophagus nearly abuts the left atrium (Circulation. 2005; 112:1400-1405; the contents of which being incorporated herein in their entirety). Both structures have courses varying from individual to individual. Imprecision in ablation in areas close to the phrenic nerve or the esophagus can lead to devastating and sometimes fatal complications.

For maximum effectiveness, pulmonary vein ablation should address all fibers in a circumferential manner. However, circumferential application of RF energy using presently available technology can lead to pulmonary vein stenosis—a serious and basically untreatable complication. It has been shown that the body's natural response to the placement of an ablation lesion is a localized proliferation of smooth muscle cells that may increase the thickness of the tissue, and hence, obstruct blood flow (a stenosis). Ablation lesions that are too concentrated present a risk of causing a concentrated proliferation of tissue resulting in clinically significant stenosis. Too obviate this problem, ablations around the pulmonary veins and the pulmonary venous antra are performed as a series of ablation points—as opposed to a continuous line—in an effort to create a pattern of lesions sufficient to cover the circumference of the vessel but diffuse enough in arrangement to prevent a stenosis. This naturally creates a balancing of the risks between incomplete lesion coverage, which may result in further AF episodes, and an overly dense lesion coverage which may result in any of stenosis, phrenic nerve damage, and/or esophageal damage. With available tools and methods, the procedure is both imprecise and time consuming, and the procedure demands a high degree of medical skill to perform. With presently used methods of RF ablation, which use imprecise temperature control, charring of blood and tissue is an additional problem. Because of the procedure length, adequate anticoagulation is difficult to maintain throughout the procedure. Both charring and blood clots can lead to stroke—both overt and silent. Incidence is approximately 1%, with silent strokes detectable by advanced MRI techniques occurring 25-30% of cases. A shortened procedure that helps to avoid negative side effects is needed.

### Similarity of Problem of Pulmonary Vein Denervation and Renal Denervation

It has been conclusively demonstrated that the nerves surrounding the renal arteries can be effectively denervated with heat energy applied from within the renal, artery lumen using either RF energy or ultrasound; and when this energy is appropriately regulated, denervation can occur without damage to the renal artery. The distribution of the nerves surrounding the pulmonary veins and those surrounding the renal arteries is similar, as is the distance of the nerves from the vessel lumen. Two dissimilarities between the systems are vessel diameters (4-8 mm for renal arteries and 5-16 mm for pulmonary veins), and differing characteristics of the vessel walls. The fact that transmission of heat energy through renal arterial walls can be done without damage to the artery does not assure that pulmonary veins would not be so damaged during transmission of the same energy. However, there is a large human experience delivering RF energy at high doses to the pulmonary veins. By using energy below a charring or vaporizing temperature, in the manner described above, damage to the veins may be avoided unless the energy is delivered in a circumferentially oriented manner. Therefore, learning from treatment methods and devices that have been successfully applied in the renal arteries is useful in providing improvements to methods and devices available to address the complex problems associated with AF ablation.

In light of the foregoing, there remains a need to provide a simple device and method of treatment to produce pulmonary vein lesions sufficient to halt AF episodes while avoiding the complications caused by tissue trauma or incomplete pulmonary vein isolation.

### SUMMARY OF THE INVENTION

The isolation of pulmonary veins in an AF ablation presents a complex set of problems for the physician. The very close proximity of the phrenic nerve and esophagus to the pulmonary veins requires that treatment energy be carefully and precisely delivered so as to avoid adversely affecting adjacent structures. However, a less than complete delivery of treatment energy can leave open a conductive path for spread of the trigger electrical impulses that cause AF. Coupled with these two problems is the additional problem that stenosis may develop in response to an overly concentrated grouping of lesions in the pulmonary veins. The present approach to this problem is to create a set of interrupted ablation points oriented circumferentially at the ostia of the pulmonary veins and around the antrum. By definition, such a set of lesions will in some cases be spaced too close together (and thus prone to complications), or too far apart (and thus not fully electrically isolating the pulmonary veins. A better solution to this set of problems is to deliver treatment energy in a pattern that covers substantially the full circumference of the pulmonary vein lumen, but where the pattern is a plurality of individual lesions that are circumferentially and axially offset so as to form a pattern that loosely approximates a helix or other staggered pattern. Such a pattern axially distributes the treatment lesions such that if some stenosis were to naturally occur in response to treatment, the overall effect of stenosis is dispersed enough to avoid a deleterious reduction of the cross section of the pulmonary vein lumen at any given point along its length.

A catheter-based expandable structure with a plurality of energy delivery surfaces is a particularly advantageous way to access the pulmonary veins. Structures with circumferentially and axially offset arrays of energy delivery surfaces may be made suitable for pulmonary vein isolation by sizing structures from approximately 5 mm to approximately 16 mm and arranging the number and location of energy delivery surfaces so as to provide loosely helical or staggered lesion patterns that cover the full circumference of the inner pulmonary vein lumen while providing axial dispersion sufficient to prevent a deleterious reduction in lumen diameter from stenosis. In many embodiments, the lesion pattern is created at a plurality of locations simultaneously during the delivery of treatment energy. In many embodiments, expandable structures have a tapered diameter where the proximal portion of the expandable structure has a larger diameter than the distal portion.

Examples of suitable catheter-based structures that may be modified to perform pulmonary denervating vein isolation in accordance with the present invention include those shown in U.S. patent application Ser. Nos. 12/206,591; 10/232,909; 11/420,419; 13/087,163; 11/782,451; 10/938,138; 11/392,231; 12/640,664; 11/420,712; 12/616,758; 13/087,163; 11/782,451; 12/700,524; 11/975,651; 11/975,474; 12/127,287; 13/562,150, the complete contents of each being incorporated herein by reference.

Another example of a balloon structure is from Vessix Vascular of Laguna Hills, Calif., which has been publicly disclosed on its website (www.vessixvascular.com) and at medical conferences, wherein a balloon catheter has surface mounted flexible circuit electrodes that deliver bipolar radiofrequency energy. The Vessix Vascular balloon catheter design can be adapted to provide a denervating pulmonary vein isolation treatment of the present invention.

Additionally, other structures such as expandable coils or probes common to current AF treatment procedures may be adapted to provide denervating energy to isolate the pulmonary veins as described by the present invention.

To achieve complete isolation of the pulmonary vein through a denervation energy treatment of the present invention, a plurality of one or more surfaces is used; the maximum number of energy delivery surfaces may be limited by size and stiffness constraints of the catheter which would be associated with the quantity of energy conductors leading from the energy delivery surfaces to the energy source. The size and spacing of energy delivery surfaces is arranged based on the desired size of the lesion created by the treatment energy dose. The denervating energy doses of the present invention are lower than the tissue vaporizing or burning energy doses of existing AF ablation treatments. Therefore a larger energy delivery surface may be employed. However, the optimized sizing of the surface is ultimately a function of the power of the energy delivered. In radiofrequency (“RF”) energy embodiments the spacing between energy delivery surfaces may range from about 0.1 mm to about 20 mm.

Structures with energy delivery surfaces may be further comprised to include one or more temperature sensing devices such as thermistors or thermocouples mounted in proximity to one or more energy delivery surfaces. Temperature sensing devices may be configured to provide feedback information to a control algorithm in a controller adapted to operate in conjunction with an energy source. Denervating pulmonary vein isolation should provide an energy treatment sufficient to denature tissues without causing tissue vaporization or charring. Therefore, a temperature-based control algorithm is a preferred method for maintaining treatment temperatures below those which would vaporize or char tissue. In addition, one or more of voltage, current, and impedance may be used as primary or secondary control algorithm factors. Embodiments of the present invention use one or more of temperature, voltage, current, and impedance as control algorithm factors to deliver energy sufficient to cause pulmonary vein isolation by denervating tissue without causing vaporization or charring. At any point before or during the application of treatment energy, an energy delivery surface may be temporarily modulated or completely deactivated if a feedback condition is outside of algorithm parameters. This approach helps to avoid the risk of coagulum formation, and phrenic nerve or esophageal damage, while providing energy sufficient to denature nerve tissue, and hence, isolate the pulmonary veins to achieve an efficacious treatment of AF while avoiding the risk of stenosis.

The temperature to achieve denervation is approximately 50 C to approximately 80 C with a treatment energy of approximately 0.25 W to approximately 100 W and with a treatment duration of approximately 10 seconds to approximately 5 minutes.

The method of access to the pulmonary veins may be by any of those used in AF treatment and catheter-based intervention including endoscopically through the wall of the heart, by a percutaneous venous approach, or by an arterial approach.

In one preferred embodiment of the present invention, a balloon catheter is positioned into the pulmonary vein such that the proximal end of the balloon is just at or slightly inside the ostium of the vein. The balloon is deployed and expanded to place the balloon in contact with the lumen of the vein. On the balloon is an array of individual flexible circuit electrodes positioned with a circumferential and axial offset from one another so as to loosely approximate a helical pattern on the surface of the balloon. The electrodes are configured to deliver bipolar RF energy. Conductors passing through the body of the catheter electrically connect the electrodes to an RF generator and controller. The electrodes are individually configured to be energized and controlled in a modulated fashion so as to precisely maintain a treatment temperature in accordance with a control algorithm. Temperature may or may not be ramped according to the treatment algorithm. The treatment energy is applied in accordance with the treatment algorithm and a denervating energy treatment is delivered to accomplish isolation of the pulmonary vein as part of an AF treatment procedure.

In another embodiment of the present invention, a balloon catheter is positioned into the pulmonary vein such that the proximal end of the balloon is just at or slightly inside the ostium of the vein. The balloon is deployed and expanded to place the balloon in contact with the lumen of the vein. On the balloon is an array of individual flexible circuit electrodes positioned with a circumferential and axial offset from one another so as to loosely approximate a helical pattern on the surface of the balloon. The electrodes are configured to deliver monopolar RF energy. A common ground may be one of the electrodes, which in turn may optionally be varied by the control algorithm so as to select different electrodes as the ground during the course of treatment, or an external grounding pad may be employed. Conductors passing through the body of the catheter electrically connect the electrodes to an RF generator and controller. The electrodes are optionally individually configured to be energized and controlled in a modulated fashion to maintain a treatment temperature in accordance with a control algorithm. The treatment energy is applied in accordance with the treatment algorithm and a denervating energy treatment is delivered to accomplish isolation of the pulmonary vein as part of an AF treatment procedure.

In another embodiment of the present invention, the balloon structure is tapered so as to have a larger diameter on its proximal end and a smaller diameter on its distal end.

In some embodiments of the present invention, a plurality of energy delivery surfaces on the balloon are distributed with a circumferential and axial offset from the immediately adjacent individual energy delivery surfaces of the plurality.

In another embodiment of the present invention, the catheter-based expandable structure is configured to include RF electrodes on a basket-like structure, which may be closed or open-ended at the basket-like structure's distal end.

In some embodiments of the present invention, the basket-like structure is tapered so as to have a larger diameter on its proximal end and a smaller diameter on its distal end.

In some embodiments of the present invention, a plurality of energy delivery surfaces on the basket-like structure are distributed with a circumferential and axial offset from the immediately adjacent individual energy delivery surfaces of the plurality.

In another embodiment of the present invention, the catheter-based expandable structure configured to include RF electrodes is a coil-like structure, which includes electrodes at points along the coil, which are positioned to create a series of energy delivery locations that loosely approximate a helical pattern as described herein.

In some embodiments, the coil-like structure is tapered so as to have a larger diameter on its proximal end and a smaller diameter on its distal end.

In another embodiment of the present invention, the catheter-based device is configured to include a probe-like structure wherein the probe is configured to include one or more RF electrodes at its distal end.

In another embodiment of the present invention, a balloon catheter is positioned into the pulmonary vein such that the proximal end of the balloon is just at or slightly inside the ostium of the vein. The balloon is deployed and expanded to place the balloon in contact with the lumen of the vein. On the balloon is an array of ultrasound transducers positioned with a circumferential and axial offset from one another so as to loosely approximate a helical pattern on the surface of the balloon. Conductors passing through the body of the catheter connect the ultrasound transducers to a generator and controller. The ultrasound transducers are optionally individually configured to be energized and controlled in a modulated fashion to maintain a treatment temperature in accordance with a control algorithm. The treatment energy is applied in accordance with the treatment algorithm and a denervating energy treatment is delivered to accomplish isolation of the pulmonary vein as part of an AF treatment procedure.

In another embodiment of the present invention, the catheter-based expandable structure is configured to include ultrasound transducers on a balloon, which is part of a catheter system having an ultrasound energy source and controller.

In some embodiments of the present invention, a plurality of ultrasound transducers on the balloon are distributed with a circumferential and axial offset from the immediately adjacent ultrasound transducers of the plurality.

In another embodiment of the present invention, the catheter-based expandable structure is configured to include ultrasound transducers on a basket-like structure, which may be closed or open-ended at the basket-like structure's distal end, and which is part of a catheter system having an ultrasound energy source and controller.

In some embodiments of the present invention, a plurality of ultrasound transducers on the basket-like structure are distributed with a circumferential and axial offset from the immediately adjacent ultrasound transducers of the plurality.

In another embodiment of the present invention, the catheter-based expandable structure is configured to include ultrasound transducers on a coil structure, which is part of a catheter system having an ultrasound energy source and controller.

In another embodiment of the present invention, the catheter-based structure is configured to include ultrasound transducers on a probe-like structure, which is part of a catheter system having an ultrasound energy source and controller.

In another embodiment of the present invention, a cryogenic source is operatively coupled to the delivery surfaces of any of the structures described herein. The delivery of the cryogen is modulated to the energy delivery surfaces according to a control algorithm and feedback as described herein so as to create an approximately helical pattern of PV-isolating lesions adjacent the energy delivery surfaces while avoiding damage to tissues such as the phrenic nerve and esophagus.

In some embodiments of the present invention, a catheter-based structure is positioned in the LA at the antrum region adjacent the right or left inferior and superior PV. An expandable structure at the distal end of the catheter is expanded to a diameter of about 3 centimeters to about 10 centimeters so as to achieve apposition of a plurality of energy delivery surfaces with the tissue of the antrum region. A lesion pattern may then be created in accordance with the energy delivery aspects of the present invention further described herein. The lesion pattern may be a plurality of individual lesions that may be annular about the circumference of the antrum, and further may optionally be offset from one another so as to create a stagger between individual lesions of the pattern, or alternately, the lesion may be substantially a single continuous lesion. The expandable structure may be a basket, a balloon, or a coil. The antral catheter system would be connected to an energy source modulated by a controller as described above. The energy source may be RF, ultrasound or cryogenic.

### BRIEF DESCRIPTION OF THE DRAWINGS

FIG. 1 shows a schematic view of the human heart with an example device embodiment accessing a pulmonary vein.

FIG. 2 shows a schematic view of balloon catheter device embodiments used in the present invention.

FIG. 2A shows a schematic view of balloon catheter device embodiments used in the present invention.

FIG. 2B shows a schematic view of balloon catheter device embodiments used in the present invention.

FIG. 3 shows a representative lesion pattern embodiment of the present invention.

FIG. 3A shows the lesion pattern of FIG. 3 unrolled about axis a-b in a flat plane.

FIG. 3B shows the lesion pattern of FIG. 3 as a sectional view about plane X-X.

FIG. 3C shows an alternate examples of lesion patterns for embodiments of the present invention.

FIG. 3D shows an alternate example of lesion patterns for embodiments of the present invention.

FIG. 4 shows a schematic sectional view of a lesion pattern and an example device embodiment of the present invention.

FIG. 4A shows a sectional view of a lesion pattern embodiment of the present invention as viewed in a circumferential cross section at a location distal from the lesion pattern looking proximally toward the pulmonary vein ostium.

FIG. 5 shows a schematic view of a closed basket structure embodiment at the distal end of a catheter device used in the present invention.

FIG. 5A shows a schematic view of an open basket structure embodiment at the distal end of a catheter device used in the present invention.

FIG. 5B shows a schematic views of an examples of a tapered basket-like structures used in the present invention.

FIG. 5C shows a schematic view of an example of a tapered basket-like structure used in the present invention.

FIG. 6 shows a schematic view of a coil structure embodiment at the distal end of a catheter device used in the present invention.

FIG. 6A shows a schematic view of a coil structure embodiment at the distal end of a catheter device used in the present invention.

FIG. 7 shows a schematic view of a probe structure embodiment at the distal end of a catheter device used in the present invention.

FIG. 8 shows a schematic view of an energy delivering catheter system embodiment used in the present invention.

FIG. 9 shows the steps of a treatment method embodiment of the present invention.

FIG. 10 shows a schematic view of an example cryogenic balloon embodiment of the present invention.

FIG. 10A shows a schematic views of an examples of a tapered basket-like structures of the present invention.

FIG. 10B shows a schematic view of an example of a tapered basket-like structure used in the present invention.

FIG. 11 shows a schematic view of the left atrium with an example of lesion formation in the antrum area of the pulmonary veins.

FIG. 12 shows a schematic view of an example embodiment of the present invention used for lesion formation in the antrum area of the pulmonary veins.

FIG. 13 shows a schematic view of a balloon catheter device embodiment used in the present invention.

FIG. 14 shows a schematic view of a coil structure embodiment at the distal end of a catheter device used in the present invention.

### DETAILED DESCRIPTION OF THE INVENTION

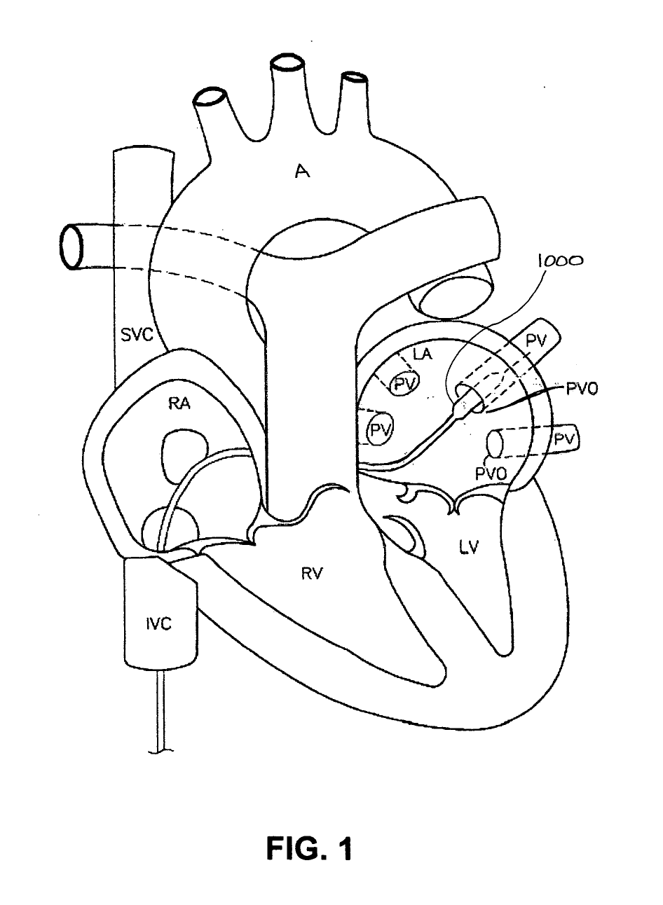

Referring to FIG. 1, the human heart is a complex hollow structure having numerous discrete sub-structures. The four chambers of the heart are the right atrium (“RA”), the right ventricle (“RV”), the left atrium (“LA”), and the left ventricle (“LV”), Several major blood vessels flow to or from the heart. The inferior and superior vena cava (“IVC” and “SVC” respectively) return blood to the heart. The aorta (“A”) supplies blood to the major portion of the body from the heart. The pulmonary veins (“PV”) provide blood from the lungs to the heart. Inside the LA are the four openings where blood from the lungs enters the LA from the PV through the pulmonary venous ostia (“PVO”). Shown is an exemplary embodiment of a balloon catheter device 1000 for use in the present invention. A venous approach to the heart through the IVC is shown. However, any of the large variety of interventional access methods used for heart procedures may be used. For example, arterial access may be used, endoscopic access may be used by a method such as by trans-apical or sub-xiphoid approach, and the like, depending on the preferences of the physician performing the AF treatment. The choice of approach may be influenced in part by the device embodiment used to create the lesion pattern of the present invention.

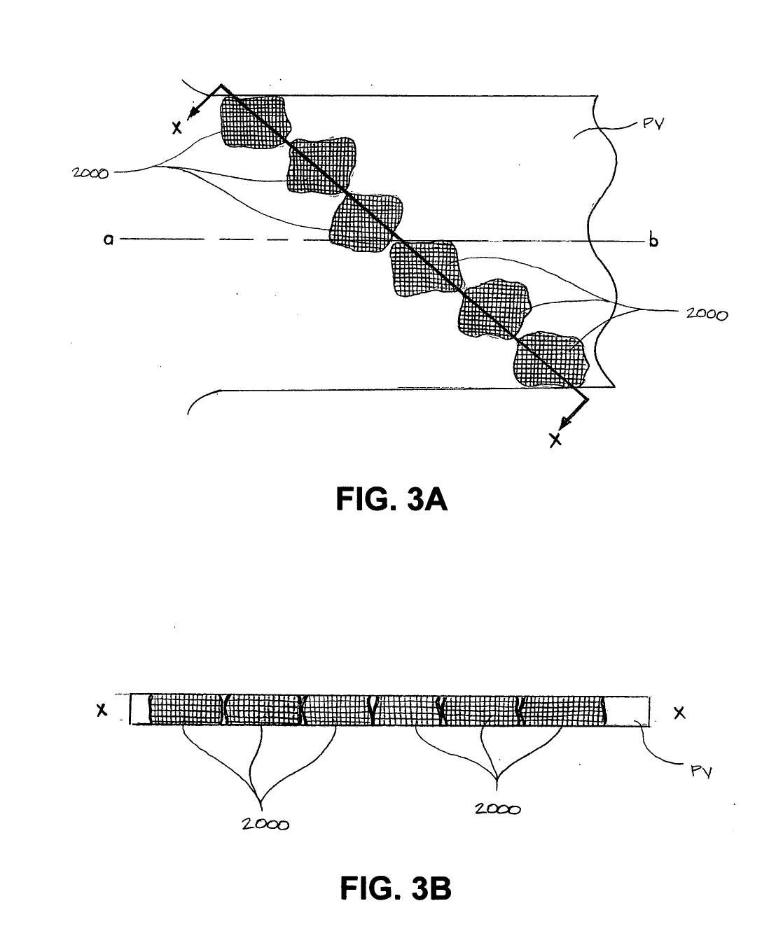

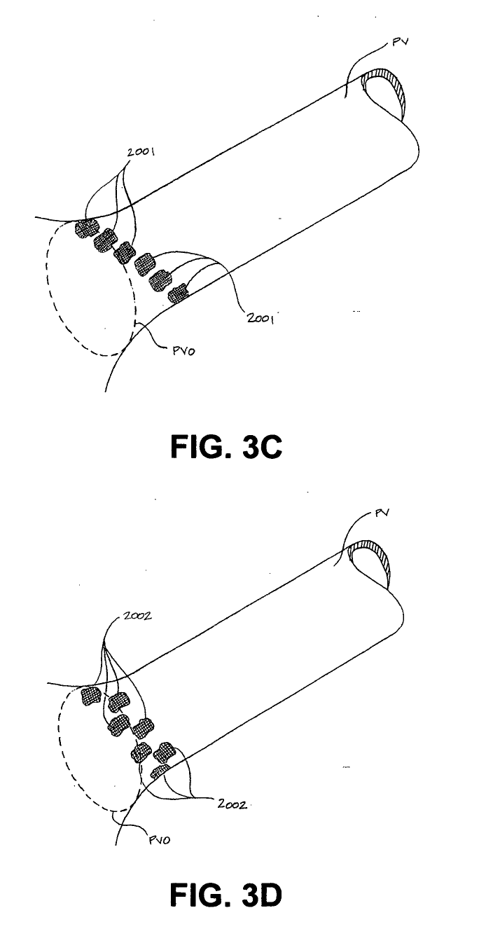

Referring to FIGS. 3, 3A, 3B, a pattern of lesions 2000 is created about the circumference and length of PV such that when viewed in a flat plane (FIG. 3A) or in a plane perpendicular to the lesion pattern (FIG. 3B), a substantially continuous pattern of lesions 2000 is formed about the circumference of PV where each of the lesions 2000 is axially offset from one another along the length of the PV.

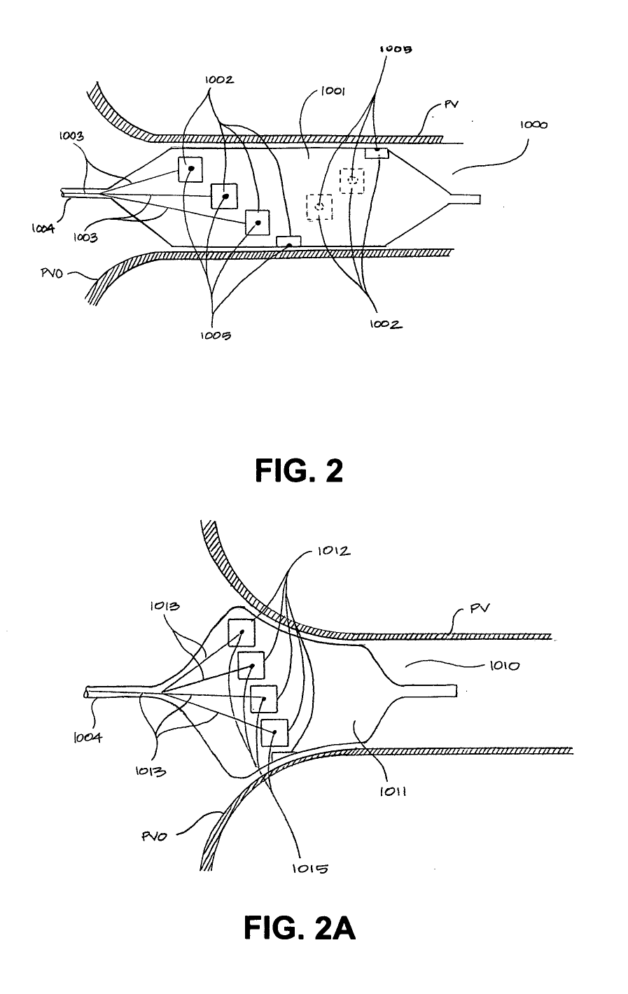

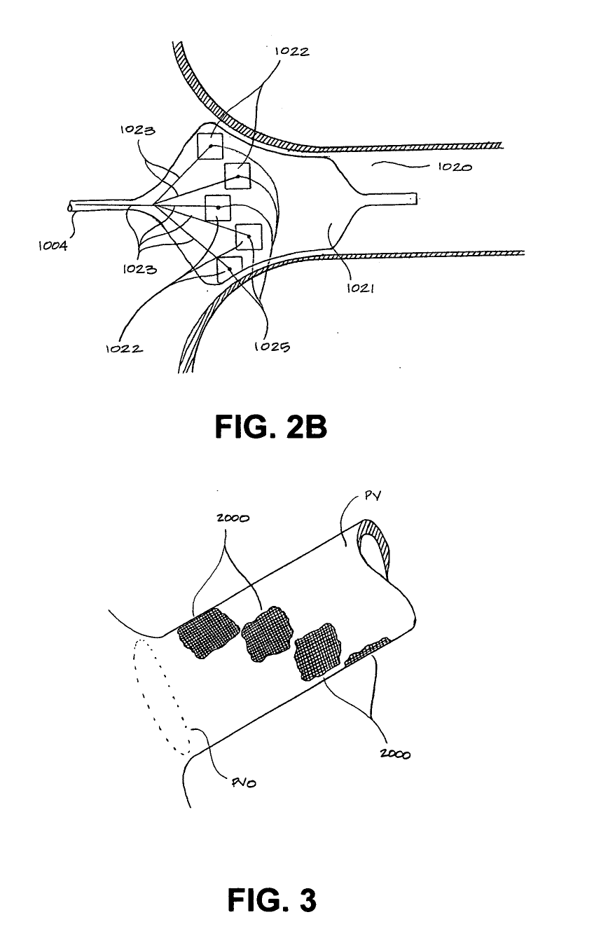

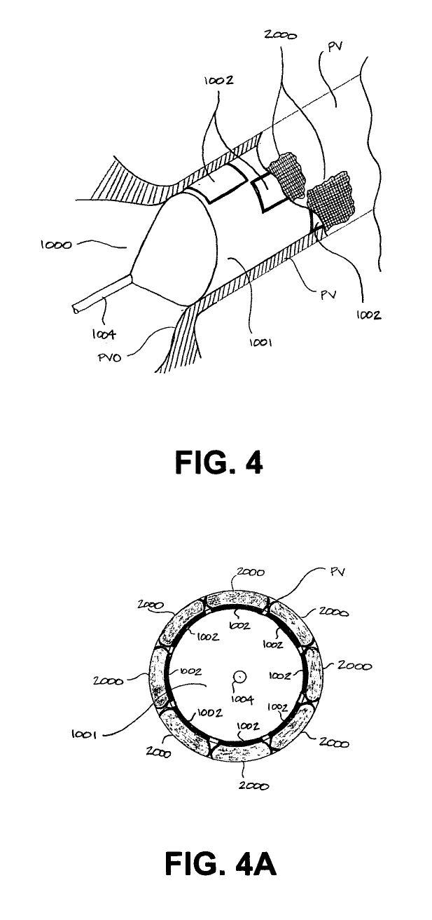

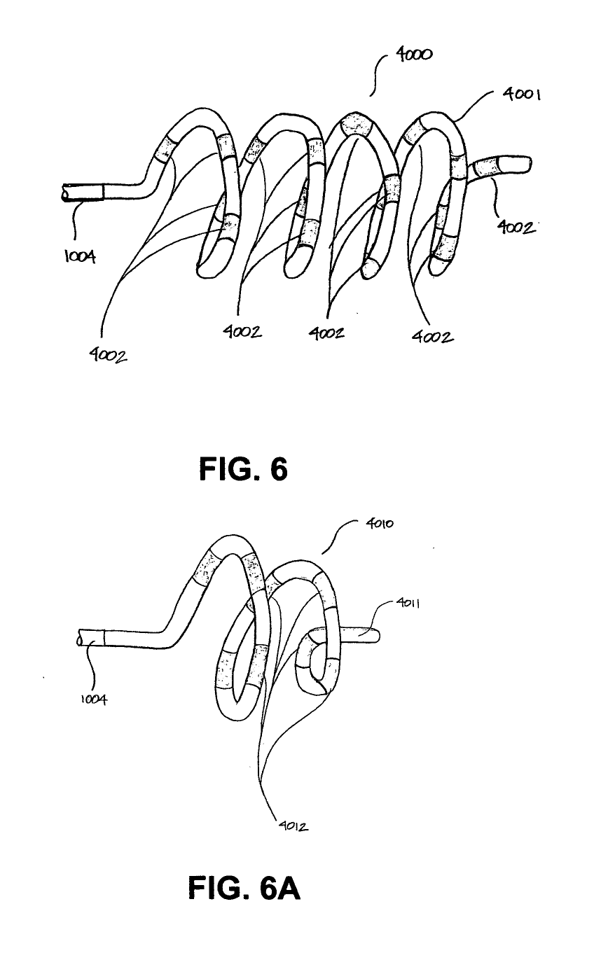

Referring now to FIG. 2, a balloon catheter device 1000 is shown having been positioned and inflated just at the PVO and inside the PV. Balloons may range in expanded diameter from about 5 mm to about 16 mm, which may further include tapered diameters with the larger diameter being at the ostial end of the balloon when placed in the PV. A plurality of energy delivery surfaces 1002 are positioned to be circumferentially and axially offset from one another on balloon 1001 so as to loosely approximate a helical pattern. Any circumferentially and axially staggered pattern may be used, the term helical being a convenient description for any staggered pattern employed. Adjacent or integrated with energy surfaces 1002, one or more optional temperature sensors 1005 may be included. Temperature sensors 1005 may be thermistors or thermocouples and may be in direct or indirect contact with tissue and/or the energy delivery surfaces 1002. Conductors 1003 run proximally through catheter body 1004 and operatively connect the energy delivery surfaces to an energy source and controller. FIG. 8 shows a catheter system 6010 with an integrated energy source and controller 6005. Catheter body 1004 (as also shown in FIGS. 2, 2A, 2B, 4, 4A, 5, 5A, 5B, 5C, 6, 6A, 7, 8, 10, 10A, and 10B) is operatively connected to power source 6005 by a connector 6004 such that conductors pass through a port 6002 of a catheter hub 6000. Catheter hub 6000 may have a guidewire and/or fluid conducting port 6003 in communication with a lumen in catheter body 1004. Catheter hub 6000 may have an inflation port 6001 in communication with lumens in catheter body 1004. The configurations of ports in catheter hub 6000 and lumens in catheter body 1004 may depend on the structural embodiment at the distal end of the catheter where the energy surfaces are located. For example, catheter body 1004 would have an inflation lumen for embodiments where a balloon is located at its distal end, while baskets, coils and probes would not require an inflation lumen but may be configured to include a lumen for guidewires, aspiration and/or perfusion. Additionally, baskets, coils or probes may include mechanical devices for deployment and/or tip deflections. A guidewire lumen would be a preferred embodiment of catheter body 1004 given that over-the-wire and rapid exchange configurations are standard in catheter-based interventional tools.

Referring to FIGS. 2A and 3C, an alternate example balloon embodiment 1010 has a tapered balloon body 1011 with diameters ranging from about 5 mm to about 16 mm, with the larger diameter being at the proximal (ostial end) of the balloon when placed in the PVO. A plurality of energy delivery surfaces 1012 are positioned to be circumferentially and axially offset from one another on balloon 1011. Adjacent or integrated with energy surfaces 1012, one or more optional temperature sensors 1015 may be included. Temperature sensors 1015 may be thermistors or thermocouples and may be in direct or indirect contact with tissue and/or the energy delivery surfaces 1012. Conductors 1013 run proximally through catheter body 1004 and operatively connect the energy delivery surfaces to an energy source and controller. A pattern of lesions 2001 is concentrated in and about the PVO.

Referring to FIGS. 2B and 3D, an alternate example balloon embodiment 1020 has a tapered balloon body 1021 with diameters ranging from about 5 mm to about 16 mm, with the larger diameter being at the proximal (ostial end) of the balloon when placed in the PVO. A plurality of energy delivery surfaces 1022 are positioned to be circumferentially and axially offset from one another on balloon 1021. Adjacent or integrated with energy surfaces 1022, one or more optional temperature sensors 1025 may be included. Temperature sensors 1025 may be thermistors or thermocouples and may be in direct or indirect contact with tissue and/or the energy delivery surfaces 1022. Conductors 1023 run proximally through catheter body 1004 and operatively connect the energy delivery surfaces to an energy source and controller. A pattern of lesions 2002 is concentrated in and about the PVO.

Referring to FIGS. 1-4A, and FIG. 8, a balloon catheter system 6010 with distal configuration 1000, is positioned into the PV such that the proximal end of a balloon (1001, 1011, 1021) is just inside the PVO. The balloon is deployed and expanded to place it in contact with the lumen of the PV.

In some embodiments, on the balloon is an array of energy delivery surfaces configured as individual flexible circuit electrodes positioned with a circumferential and axial offset from one another so as to loosely approximate a helical or staggered pattern on the surface of balloon. The electrodes are configured to deliver bipolar RF energy. Conductors passing through catheter body 1004 electrically connect the electrodes to an RF generator and controller 6005 via a catheter hub 6000 and an electrical connector 6004. The electrodes (1002, 1012, 1022) are individually configured to be energized and controlled in a modulated fashion so as to precisely maintain a treatment temperature in accordance with a control algorithm programmed in the software memory of controller 6005. The treatment energy is applied in accordance with the treatment algorithm and a denervating energy treatment is delivered to accomplish isolation of PV by creating a pattern of lesions corresponding to the position of the electrodes. The resultant pattern of lesions is distributed at discrete locations about the circumference and length of PV, and when viewed in a plane perpendicular to the length of PV cover substantially the complete circumference of PV.

The denervating energy treatment is applied in the form of a mild heating of tissue, which avoids the deleterious damaging effects of tissue vaporization or tissue charring by delivering energy as a therapeutic dose. A denervating energy treatment is sufficient to cause the denaturing of targeted tissue while applying energy at a level that avoids thermally damaging adjacent tissue. The temperature range at which this occurs is from about 50 C to about 80 C. In this range, the conductive nerve tissue in the wall of the PV undergoes cellular necrosis while avoiding the gross tissue trauma, and resultant cellular proliferation, that results from vaporization or charring.

The control algorithm for generator 6005 may detect contact with tissue by sensing impedance levels at the electrodes. The algorithm selectively energizes electrodes when the treatment is initiated. Individual control of electrodes may be accomplished by modulating a time and/or level of powering in accordance with the control algorithm and feedback sensed at the electrodes and/or temperature sensors. The algorithm may use any of temperature, voltage, current, and impedance, or any combination thereof, as control variables in the algorithm. The application of bipolar RF energy during the course of a treatment ranges from approximately 0.25 W to approximately 25 W of power for a total treatment time from approximately 10 seconds to approximately 2 minutes. During the application of energy, the control algorithm senses whether the control variables are within defined limits according to the software program and feedback. When a variable is outside of its limits, the energy applied to an individual electrode is modulated by increasing, decreasing, or halting applied energy in accordance with the limits of the algorithm equation and during the segment of cycle time for which the modulation condition exists (such as microseconds, milliseconds, seconds). This control method is applied over the course of the treatment period until the treatment endpoint is reached. The treatment endpoint may be any one or more of time, temperature, and impedance. The energy dosage necessary to achieve an efficacious denervation varies by the type of body lumen involved and the energy delivery surface configuration being used. In the case of a PV isolation procedure, the PV ranges in diameter from about 5 mm to about 16 mm and the PV is heavily perfused with blood. As compared to delivery of energy in a peripheral vessel or delivery of energy in a renal artery, energy delivery surfaces may be larger in size and/or higher in number in order to provide the necessary lesion pattern while seeking to preserve a mild heating that avoids charring, stenosis, phrenic nerve damage, or esophageal damage.

Referring again to FIGS. 1-4A, and FIG. 8, another embodiment of the present invention, energy delivery surfaces (1002, 1012, 1022) are electrodes configured to deliver monopolar RF energy. A common ground may be one of the electrodes, which in turn may optionally be varied by the control algorithm so as to select different electrodes as the ground during cycle time periods over the course of treatment, or an external grounding pad (not shown) may be employed. Conductors passing through catheter body 1004 electrically connect the electrodes to a RF generator and controller 6005 via a catheter hub 6000 and an electrical connector 6004. The electrodes are optionally individually configured to be energized and controlled in a modulated fashion to maintain a treatment temperature in accordance with a control algorithm programmed in the software memory of controller 6005. The treatment energy is applied in accordance with the treatment algorithm and a denervating energy treatment is delivered to accomplish isolation of PV by creating a pattern of lesions corresponding to the position of the electrodes. The resultant pattern of lesions is distributed at discrete locations about the circumference and length of PV, and when viewed in a plane perpendicular to the length of PV cover substantially the complete circumference of PV. The application of monopolar RF energy during the course of a treatment ranges from approximately 0.25 W to approximately 100 W of power for a total treatment time of up to approximately 5 minutes.

In another embodiment, the energy delivery surfaces (1002, 1012, 1022) on the balloon (1001, 1011, 1021) are an array of ultrasound transducers. Ultrasound transducers are optionally individually configured to be energized and controlled in a modulated fashion to maintain a treatment temperature in accordance with a control algorithm. The ultrasound transducers may produce focused or unfocused ultrasound.

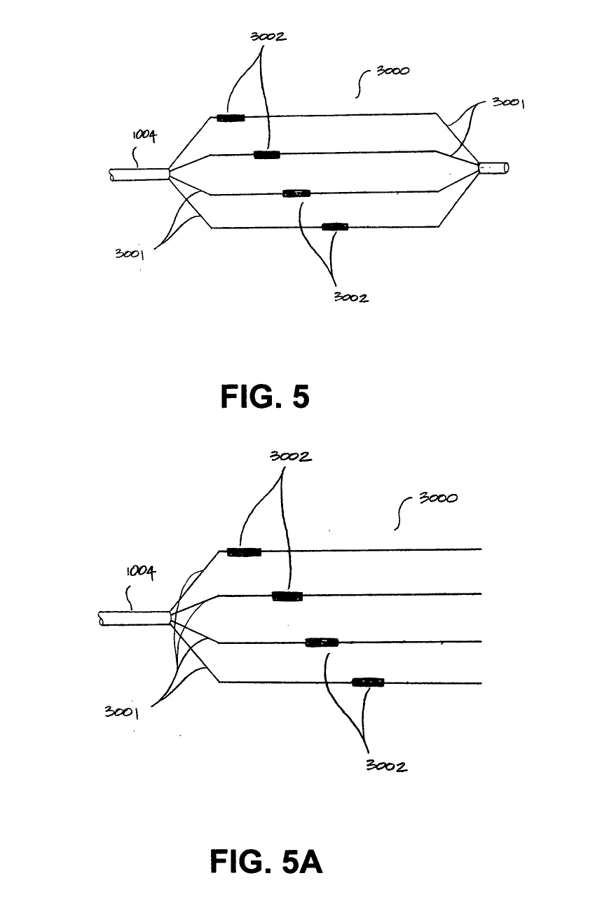

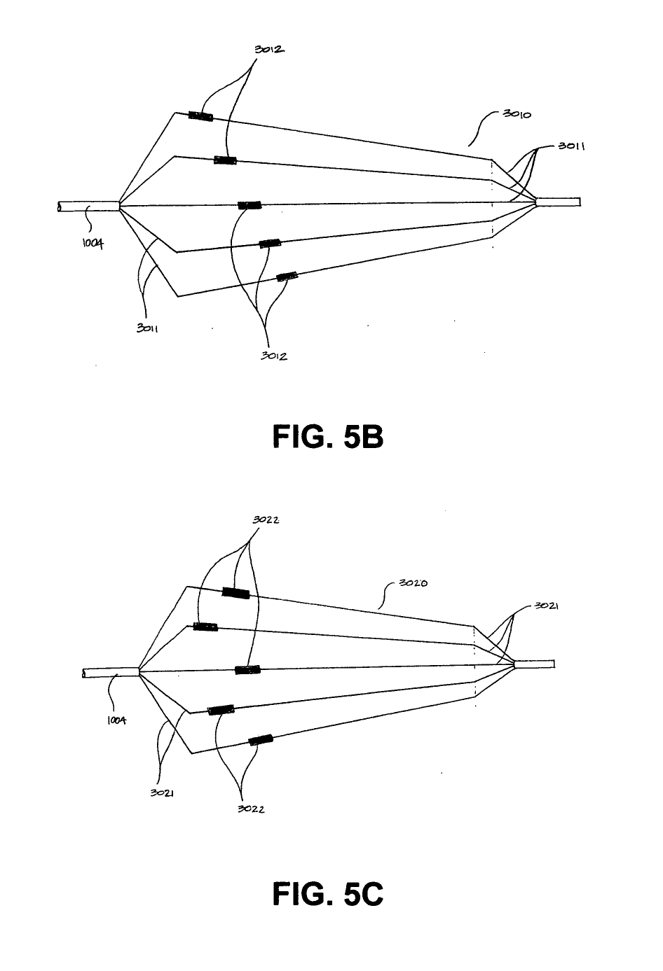

Referring to FIGS. 1, 3, 3A, 3B, 3C, 3D, 4A, 5, 5A, 5B, 5C, and 8, the catheter-based system 6010 is configured with a basket-like expandable structure (3000, 3010, 3020) at the distal end of catheter body 1004 which may range in expanded diameter from about 5 mm to about 16 mm. Optionally, the basket structure may be open on its distal end, an example of which is shown in FIG. 5A. The expandable structure has a plurality of struts (3001, 3011, 3021) that expand when deployed either by mechanical means, such as a pull wire, or by making struts from a shape memory/superelastic material such as nickel-titanium. However, the illustrated means of expanding the basket are by way of example rather than by limitation. In any basket embodiment of the present invention, the broad variety of means for actuation commonly known in the art may be used; for example a sliding collar, a retraction mechanism axially foreshortening the struts of the basket, and the like, may be used to cause the struts of the basket to open by mechanical actuation. Similarly, in basket embodiments employing shape memory/superelastic materials, any of the variety of medically suitable metals or polymers may be used. Mounted on the struts is an array of energy delivery surfaces (3002, 3012, 3022).

In some embodiments, energy delivery surfaces are configured as individual flexible electrodes positioned with a circumferential and axial offset from one another so as to loosely approximate a helical or staggered pattern on the surface of the basket structure. Adjacent or integrated with the energy delivery surfaces, one or more optional temperature sensors may be included. Temperature sensors may be thermistors or thermocouples and may be in direct or indirect contact with tissue and/or the energy delivery surfaces.

An alternate example basket embodiment 3010 has a tapered body of struts 3011, the basket diameters ranging from about 5 mm to about 16 mm, with the larger diameter being at the proximal (ostial end) of the basket when placed in the PVO. A plurality of energy delivery surfaces 3012 are positioned to be circumferentially and axially offset from one another on the basket struts 3011. Adjacent or integrated with energy surfaces 3012, one or more optional temperature sensors (not shown) may be included. Temperature sensors may be thermistors or thermocouples and may be in direct or indirect contact with tissue and/or the energy delivery surfaces 3012. Conductors run proximally through catheter body 1004 and operatively connect the energy delivery surfaces to an energy source and controller. A pattern of lesions 2001 is concentrated in and about the PVO.

Another alternate example basket embodiment 3020 has a tapered body of struts 3021, the basket diameters ranging from about 5 mm to about 16 mm, with the larger diameter being at the proximal (ostial end) of the basket when placed in the PVO. A plurality of energy delivery surfaces 3022 are positioned to be circumferentially and axially offset from one another on basket struts 3021. Adjacent or integrated with energy surfaces 302, one or more optional temperature sensors (not shown) may be included. Temperature sensors may be thermistors or thermocouples and may be in direct or indirect contact with tissue and/or the energy delivery surfaces 3022. Conductors run proximally through catheter body 1004 and operatively connect the energy delivery surfaces to an energy source and controller. A pattern of lesions 2002 is concentrated in and about the PVO.

In some embodiments, the electrodes are configured to deliver bipolar RF energy. Conductors (not shown) passing through catheter body 1004 electrically connect the electrodes to a RF generator and controller 6005 via a catheter hub 6000 and an electrical connector 6004. The electrodes are individually configured to be energized and controlled in a modulated fashion so as to precisely maintain a treatment temperature in accordance with a control algorithm programmed in the software memory of controller 6005. The treatment energy is applied in accordance with the treatment algorithm and a denervating energy treatment is delivered to accomplish isolation of PV by creating a pattern of lesions corresponding to the position of the electrodes. The resultant pattern of lesions (2000, 2001, 2002) is distributed at discrete locations about the circumference and length of PV, and when viewed in a plane perpendicular to the length of PV cover substantially the complete circumference of PV.

The denervating energy treatment is applied in the form of a mild heating of tissue which avoids the deleterious damaging effects of tissue vaporization or tissue charring by delivering energy as a therapeutic dose. A denervating energy treatment is sufficient to cause the denaturing of targeted tissue while applying energy at a level that avoids thermally damaging adjacent tissue. The temperature range at which this occurs is from about 50 C to about 80 C. In this range, the conductive nerve tissue in the wall of the PV undergoes cellular necrosis while avoiding the gross tissue trauma, and resultant cellular proliferation that results, from vaporization or charring.

The control algorithm for generator 6005 may detect contact with tissue by sensing impedance levels at electrodes. The algorithm selectively energizes electrodes when the treatment is initiated. Individual control of electrodes may be accomplished by modulating a time and/or level of power in accordance with the control algorithm and feedback sensed at the electrodes and/or temperature sensors. The algorithm may use any of temperature, voltage, current, and impedance, or any combination thereof, as control variables in the algorithm. The application of bipolar RF energy during the course of a treatment ranges from approximately 0.25 W to approximately 25 W of power for a total treatment time from approximately 10 seconds to approximately 2 minutes. During the application of energy, the control algorithm senses whether the control variables are within defined limits according to the software program and feedback. When a variable is outside of its limits, the energy applied to an individual electrode is modulated by increasing, decreasing, or halting applied energy in accordance with the limits of the algorithm equation and during the segment of cycle time for which the modulation condition exists (such as microseconds, milliseconds, seconds). This control method is applied over the course of the treatment period until the treatment endpoint is reached. The treatment endpoint may be any one or more of time, temperature, and impedance. The energy dosage necessary to achieve an efficacious denervation varies by the type of body lumen involved and the energy delivery surface configuration being used. In the case of a PV isolation procedure, the PV ranges in diameter from about 5 mm to about 16 mm and the PV is heavily perfused with blood. As compared to delivery of energy in a peripheral vessel or delivery of energy in a renal artery, energy delivery surfaces may be larger in size and/or higher in number in order to provide the necessary lesion pattern while seeking to preserve a mild heating that avoids stenosis, phrenic nerve damage, or esophageal damage.

Alternately, energy delivery surfaces may be configured to be electrodes delivering monopolar RF energy. A common ground may be one of the electrodes, which in turn may optionally be varied by the control algorithm so as to select different electrodes as the ground during cycle time periods over the course of treatment, or an external grounding pad (not shown) may be employed. Conductors (not shown) passing through catheter body 1004 electrically connect the electrodes to an RF generator and controller 6005 via a catheter hub 6000 and an electrical connector 6004. The electrodes are optionally individually configured to be energized and controlled in a modulated fashion to maintain a treatment temperature in accordance with a control algorithm programmed in the software memory of controller 6005.

In an additional monopolar electrode configuration, the struts (3001, 3011, 3021) may themselves be conductive and areas adjacent to electrode (3002, 3012, 3022) surfaces on struts are insulated from conducting energy to tissue of the PV.

The application of monopolar RF energy during the course of a treatment ranges from approximately 0.25 W to approximately 100 W of power for a total treatment time of up to approximately 5 minutes.

In another embodiment, the energy delivery surfaces (3002, 3012, 3022) on struts (3001, 3011, 3021) are an array of ultrasound transducers. Ultrasound transducers are optionally individually configured to be energized and controlled in a modulated fashion to maintain a treatment temperature in accordance with a control algorithm. The ultrasound transducers may produce focused or unfocused ultrasound.

Referring now to FIGS. 1, 3, 3A, 3B, 3C, 3D, 4A, 6, 6A, and 8, in an embodiment of the present invention, the catheter-based system 6010 is configured with a coil-like expandable structure 4000 at the distal end of catheter body 1004 ranging in expanded diameter from about 5 mm to about 16 mm, which includes energy delivery surfaces 4002 at points along the body 4001 of the coil, and which are positioned to create a series of energy delivery locations that loosely approximate a helical or staggered pattern as described herein. Adjacent or integrated with energy surfaces 4002, one or more optional temperature sensors may be included. Temperature sensors may be thermistors or thermocouples and may be in direct or indirect contact with tissue and/or the energy delivery surfaces 4002.

An alternate example coil embodiment 4010 has a body 4011, wound in a tapering coil diameter ranging from about 5 mm to about 16 mm, with the larger diameter being at the proximal (ostial end) of the coil when placed in the PVO. A plurality of energy delivery surfaces 4012 are positioned to be circumferentially and axially offset from one another on the body 4011. Adjacent or integrated with energy surfaces 4012, one or more optional temperature sensors (not shown) may be included. Temperature sensors may be thermistors or thermocouples and may be in direct or indirect contact with tissue and/or the energy delivery surfaces 4012.

In some embodiments, the energy delivery surfaces (4002, 4012) are electrodes configured to deliver bipolar RF energy. Conductors (not shown) passing through catheter body 1004 electrically connect the electrodes to an RF generator and controller 6005 via a catheter hub 6000 and an electrical connector 6004. The electrodes are individually configured to be energized and controlled in a modulated fashion so as to precisely maintain a treatment temperature in accordance with a control algorithm programmed in the software memory of controller 6005. The treatment energy is applied in accordance with the treatment algorithm and a denervating energy treatment is delivered to accomplish isolation of PV by creating a pattern of lesions (2000, 2001) corresponding to the position of the electrodes. The resultant pattern of lesions is distributed at point locations about the circumference and length of PV, and when viewed in a plane perpendicular to the length of PV cover substantially the complete circumference of PV.

The control algorithm for generator 6005 may detect contact with tissue by sensing impedance levels at electrodes. The algorithm selectively energizes electrodes when the treatment is initiated. Individual control of electrodes may be accomplished by modulating a time and/or level of powering in accordance with the control algorithm and feedback sensed at the electrodes and/or temperature sensors. The algorithm may use any of temperature, voltage, current, and impedance as control variables in the algorithm. The application of bipolar RF energy during the course of a treatment ranges from approximately 0.25 W to approximately 25 W of power for a total treatment time from approximately 10 seconds to approximately 2 minutes. During the application of energy, the control algorithm senses whether the control variables are within defined limits according to the software program and feedback. When a variable is outside of its limits, the energy applied to an individual electrode is modulated by increasing, decreasing, or halting applied energy in accordance with the limits of the algorithm equation and during the segment of cycle time for which the modulation condition exists (such as microseconds, milliseconds, seconds). This control method is applied over the course of the treatment period until the treatment endpoint is reached. The treatment endpoint may be any one or more of time, temperature, and impedance. The energy dosage necessary to achieve an efficacious denervation varies by the type of body lumen involved and the energy delivery surface configuration being used. In the case of a PV isolation procedure, the PV ranges in diameter from about 5 mm to about 16 mm and the PV is heavily perfused with blood. As compared to delivery of energy in a peripheral vessel or delivery of energy in a renal artery, energy delivery surfaces may be larger in size and/or higher in number in order to provide the necessary lesion pattern while seeking to preserve a mild heating that avoids stenosis, phrenic nerve damage, or esophageal damage.

Alternately, energy delivery surfaces (4002, 4012) may be electrodes configured to deliver monopolar RF energy. A common ground may be one of the electrodes, which in turn may optionally be varied by the control algorithm so as to select different electrodes as the ground during cycle time periods over the course of treatment, or an external grounding pad (not shown) may be employed. Conductors (not shown) passing through catheter body 1004 electrically connect the electrodes to a RF generator and controller 6005 via a catheter hub 6000 and an electrical connector 6004. The electrodes are optionally individually configured to be energized and controlled in a modulated fashion to maintain a treatment temperature in accordance with a control algorithm programmed in the software memory of controller 6005.

In an additional monopolar electrode configuration, the coil body (4001, 4011) may itself be conductive and the spaces between electrode surfaces on the coil body are insulated from conducting energy to tissue of the PV.

The application of monopolar RF energy during the course of a treatment ranges from approximately 0.25 W to approximately 100 W of power for a total treatment time of up to approximately 5 minutes.

In another embodiment, the energy delivery surfaces (4002, 4012) on the coil body (4001, 4011) are an array of ultrasound transducers. Ultrasound transducers 4002 are optionally individually configured to be energized and controlled in a modulated fashion to maintain a treatment temperature in accordance with a control algorithm. The ultrasound transducers 4002 may produce focused or unfocused ultrasound.

Referring now to FIGS. 1, 3, 3A, 3B, 3C, 3D, 4A, 7, and 8, in an embodiment of the present invention, the catheter-based system 6010 is configured with a steerable probe-like structure 5000 at the distal end of catheter body 1004, which includes energy delivery surface 5002 at points along the body 5001 of the probe. Probe body 5001 may be deflected via a control wire (not shown) to deflect the probe body 5001 and energy delivery surface 5002 to any angle up to approximately 90 degrees from the undeflected position. Adjacent or integrated with energy delivery surface 5002, an optional temperature sensor may be included. The temperature sensor may be a thermistor or a thermocouple and may be in direct or indirect contact with tissue and/or the energy delivery surface 5002. The energy delivery surface 5002 is an electrode configured to deliver bipolar RF energy. Conductors (not shown) passing through catheter body 1004 electrically connect the electrode 5002 to a RF generator and controller 6005 via a catheter hub 6000 and an electrical connector 6004. The electrode 5002 is configured to be energized and controlled in a modulated fashion so as to precisely maintain a treatment temperature in accordance with a control algorithm programmed in the software memory of controller 6005. The treatment energy is applied in accordance with the treatment algorithm and a denervating energy treatment is delivered to accomplish isolation of PV by creating in series a pattern of lesions (2000, 2001, 2002). The resultant pattern of lesions is distributed at point locations about the circumference and length of PV and/or PVO, and when viewed in a plane perpendicular to the length of PV cover substantially the complete circumference of PV and/or PVO.

Alternately, electrode 5002 may be configured to deliver monopolar RF energy. A ground may be located on probe body 5001 proximal to electrode 5002, or an external grounding pad (not shown) may be employed. Conductors (not shown) passing through catheter body 1004 electrically connect the electrode 5002 to a RF generator and controller 6005 via a catheter hub 6000 and an electrical connector 6004.

The control algorithm for generator 6005 may detect contact with tissue by sensing impedance levels at electrode 5002. The algorithm energizes electrode 5002 when the treatment is initiated. Control of electrode 5002 may be accomplished by modulating a time and/or level of powering in accordance with the control algorithm and feedback sensed at electrode 5002 and/or temperature sensors. The algorithm may use any of temperature, voltage, current, and impedance, or any combination thereof, as control variables in the algorithm. The application of energy during the course of a bipolar RF treatment ranges from approximately 0.25 W to approximately 25 W of power for a total treatment time from approximately 10 seconds to approximately 2 minutes. The application of monopolar RF energy during the course of a treatment ranges from approximately 0.25 W to approximately 100 W of power for a total treatment time of up to approximately 5 minutes. During the application of energy, the control algorithm senses whether the control variables are within defined limits according to the software program and feedback. When a variable is outside of its limits, the energy applied to electrode 5002 is modulated by increasing, decreasing, or halting applied energy in accordance with the limits of the algorithm equation and during the segment of cycle time for which the modulation condition exists (such as microseconds, milliseconds, seconds). This control method is applied over the course of the treatment period until the treatment endpoint is reached. The treatment endpoint may be any one or more of time, temperature, and impedance.

In another embodiment, the energy delivery surface 5002 on probe body 5001 is an ultrasound transducer controlled in a modulated fashion to maintain a treatment temperature in accordance with a control algorithm. The ultrasound transducer 5002 may produce focused or unfocused ultrasound.

Referring again to each of the FIGS. 1-8, generator 6005 is configured as a cryogenic source. Control of energy delivery surfaces may be accomplished by modulating a time and/or level of cryogenic delivery in accordance with the generator 6005 control algorithm and feedback sensed at cryogenic delivery surfaces and/or temperature sensors. The algorithm may use any of temperature, voltage, current, and impedance, cryogen flow rate, cryogen flow time, or any combination thereof, as control variables in the algorithm. The application of energy during the course of a treatment is based on the thermal properties of the specific cryogen being used, any of the now known cryogens for use in AF therapies being suitable, for a total treatment time from approximately 10 seconds or more. In cryogenic embodiments of the present invention, tissue treatment temperatures are below 0 C (as opposed to approximately 50 C to approximately 80 C in non-cryogenic embodiments). During cryogenic delivery, the control algorithm senses whether the control variables are within defined limits according to the software program and feedback. When a variable is outside of its limits, the cryogenic delivery applied to energy delivery surfaces is modulated by increasing, decreasing, or halting applied cryogenic delivery in accordance with the limits of the algorithm equation and during the segment of cycle time for which the modulation condition exists (such as microseconds, milliseconds, seconds). This control method is applied over the course of the treatment period until the treatment endpoint is reached. The treatment endpoint may be any one or more of time, temperature, and impedance.

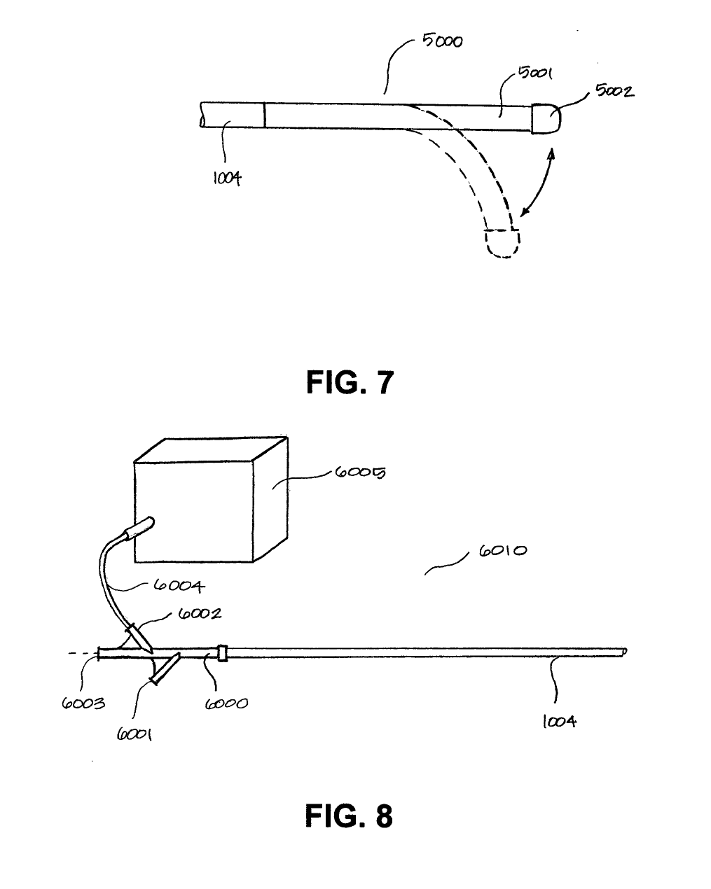

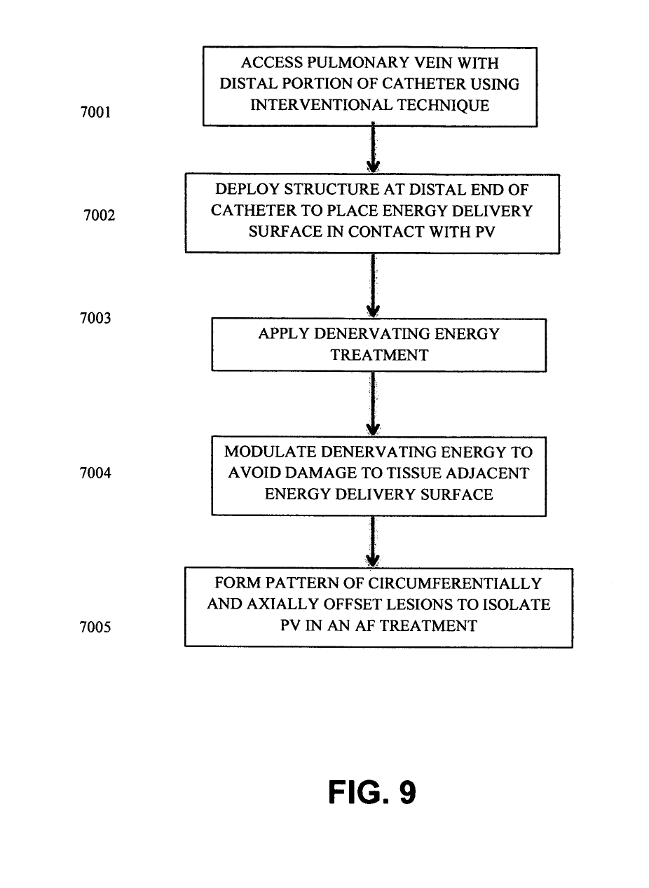

For example, the balloon 1001 of FIG. 2 may be configured to have energy delivery surfaces 1002 operatively coupled to generator 6005, which supplies a cryogen. In an alternate example, probe structure 5000 of FIG. 7 may be configured to have energy delivery surfaces 5002 operatively coupled to generator 6005, which supplies a cryogen.

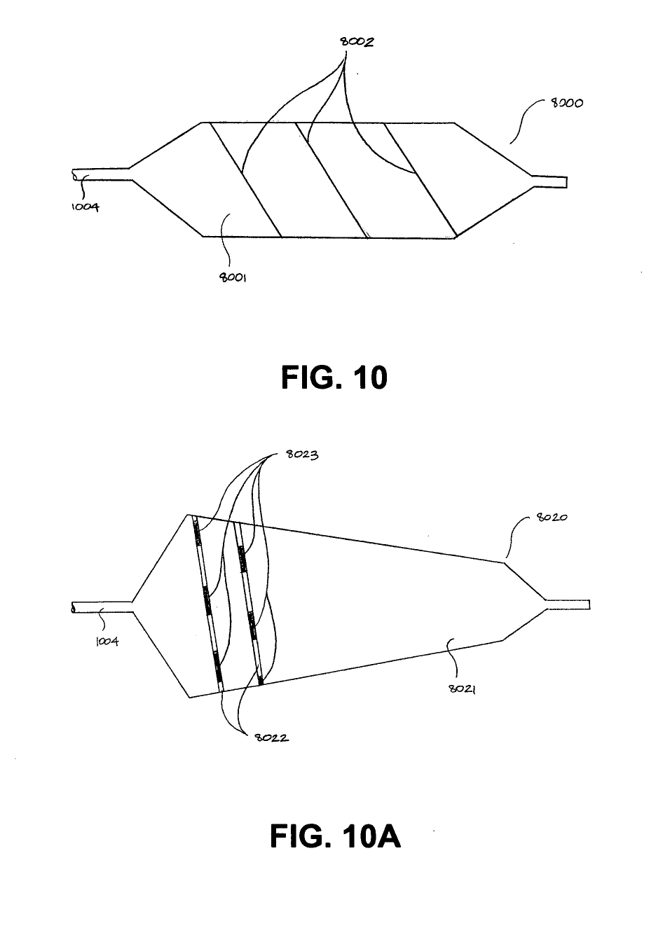

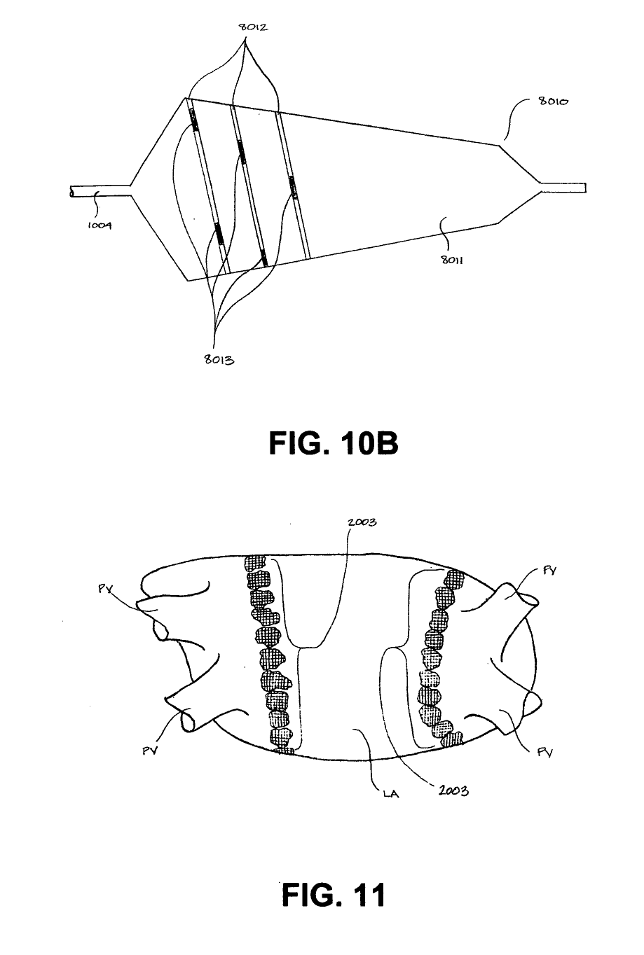

Referring to FIG. 10, an example of a cryogenic balloon structure 8000 is shown. An expandable and collapsible balloon 8001 is located at the distal end of catheter body 1004 with one or more cryogenic delivery surfaces 8002. The cryogenic delivery surfaces 8002 may be positioned either on the outer surface or the inner surface of balloon 8001. The cryogenic delivery surfaces 8002 are tubular in nature so as to conduct the cryogen through a fluid transmitting lumen, with a hypotube construction being an example of a cryogenic delivery surface 8002. Optionally, portions of the cryogenic delivery surfaces 8002 may be insulated to allow for focused delivery of treatment energy at lesion locations in a pattern of discrete locations that loosely approximate a helical pattern.

Referring to FIGS. 10B and 3C, an alternate example balloon embodiment 8010 has a tapered balloon body 8011 with diameters ranging from about 5 mm to about 16 mm, with the larger diameter being at the proximal (ostial end) of the balloon when placed in the PVO. A plurality of cryogenic delivery surfaces 8013 are positioned to be circumferentially and axially offset from one another on the hypotube 8012. A pattern of lesions 2001 is concentrated in and about the PVO. Cryogenic delivery surfaces 8013 are exposed uninsulated portions of the hypotube 8012, while the remainder of the hypotube 8012 on the balloon body 8011 is insulated thereby creating focused areas of cryogenic delivery.

Referring to FIGS. 10A and 3D, an alternate example balloon embodiment 8020 has a tapered balloon body 8021 with diameters ranging from about 5 mm to about 16 mm, with the larger diameter being at the proximal (ostial end) of the balloon when placed in the PVO. A plurality of cryogenic delivery surfaces 8023 are positioned to be circumferentially and axially offset from one another on hypotube 8022. A pattern of lesions 2002 is concentrated in and about the PVO. Cryogenic delivery surfaces 8022 are exposed uninsulated portions of the hypotube 8022, while the remainder of the hypotube 8022 on the balloon body 8021 is insulated thereby creating focused areas of cryogenic delivery.

Referring again to FIG. 7, in another embodiment, the energy delivery surface 5002 on probe body 5001 is cryogenic delivery system controlled in a modulated fashion to maintain a treatment temperature in accordance with a control algorithm.

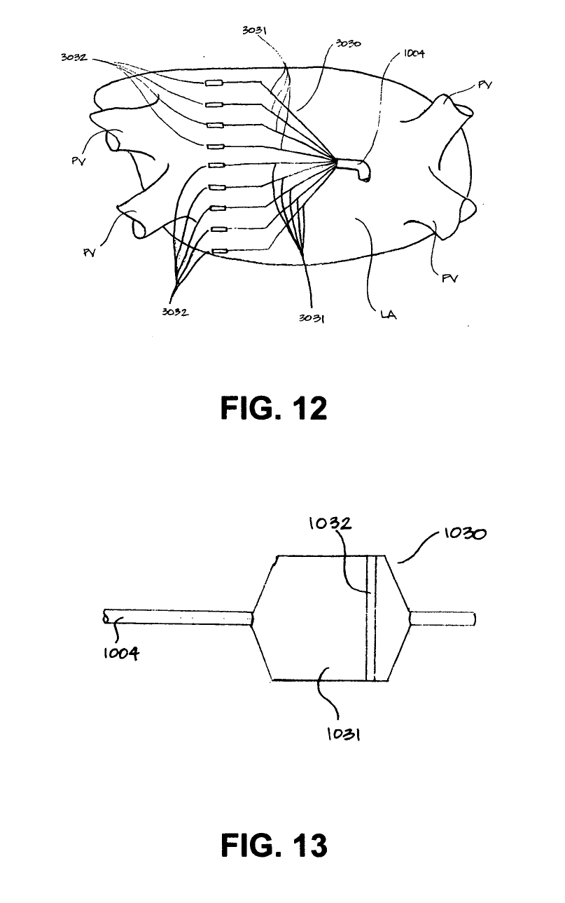

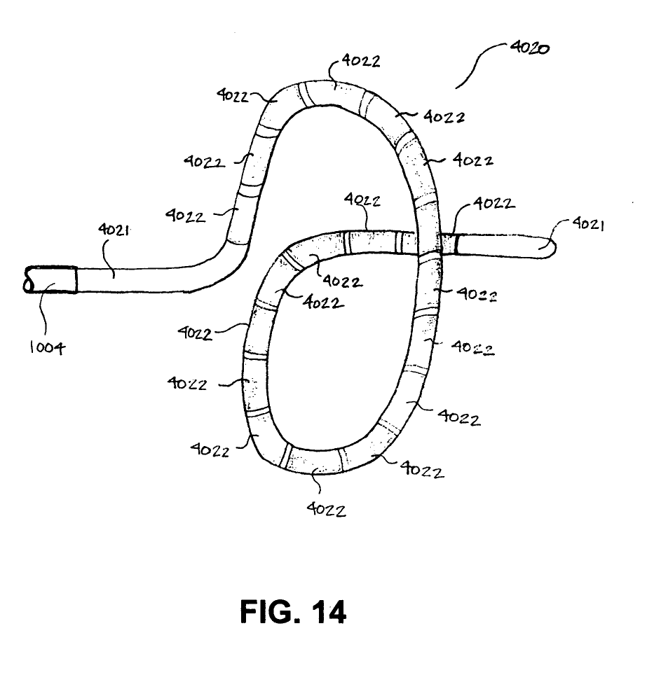

Referring now to FIGS. 11 and 12, lesions 2003 may be created about the inner circumference of the LA in proximity to the right or left PV's. In this way, isolation of the individual PV's may be obtained more rapidly than when isolating each PV individually. Moreover, the tissue of the antrum region is less prone to the risk of stenosis and is highly perfused by blood, which makes PV isolation in the antrum region an attractive means for treating AF. Lesions 2003 may be a plurality of individual lesions that are placed in an annular fashion about the circumference of the antrum in a substantially continuous form. Optionally, individual lesions of the plurality may be staggered relative to one another so that the annular form of the lesion is not perfectly contained in one cross sectional plane of the LA. In some embodiments, lesions 2003 may be a continuous lesion substantially contained within one cross sectional plane of the LA.

An example of an expandable catheter-based structure suitable for forming lesions 2003 is a basket-like structure 3030 at the distal end of catheter body 1004 which may range in expanded diameter from about 3 cm to about 10 cm. The basket may be open-ended or closed-ended, an open-ended basket is shown. The expandable structure has a plurality of struts 3031 that expand when deployed either by mechanical means such as a pull wire or by making struts from a shape memory/superelastic material such as nickel-titanium. However, the means of expanding the basket are by way of example rather than by limitation. In any basket embodiment of the present invention, the broad variety of means for actuation commonly known in the art may be used; for example a sliding collar, a retraction mechanism axially foreshortening the struts of the basket, and the like, may be used to cause the struts of the basket to open my mechanical actuation. Similarly, in basket embodiments employing shape memory/superelastic materials, any of the variety of medically suitable metals or polymers may be used. Mounted on the struts is an array of energy delivery surfaces 3032.

In some embodiments, energy delivery surfaces 3032 are configured as individual flexible electrodes positioned with a circumferential and axial offset from one another so as to loosely approximate a helical or staggered pattern on the surface of the basket structure. Adjacent or integrated with the energy delivery surfaces, one or more optional temperature sensors may be included. Temperature sensors may be thermistors or thermocouples and may be in direct or indirect contact with tissue and/or the energy delivery surfaces.

Now referring to FIGS. 8, 11, and 12, in some embodiments, the electrodes are configured to deliver bipolar RF energy. Conductors (not shown) passing through catheter body 1004 electrically connect the electrodes to an RF generator and controller 6005 via a catheter hub 6000 and an electrical connector 6004. The electrodes are individually configured to be energized and controlled in a modulated fashion so as to precisely maintain a treatment temperature in accordance with a control algorithm programmed in the software memory of controller 6005. The treatment energy is applied in accordance with the treatment algorithm and a denervating energy treatment is delivered to accomplish isolation of PV by creating a pattern of lesions corresponding to the position of the electrodes. The resultant pattern of lesions 2003 is distributed at discrete locations about the circumference of the antrum region of the LV, covering substantially the complete circumference of antrum.

The denervating energy treatment is applied in the form of a mild heating of tissue which avoids the deleterious damaging effects of tissue vaporization or tissue charring by delivering energy as a therapeutic dose. A denervating energy treatment is sufficient to cause the denaturing of targeted tissue while applying energy at a level that avoids thermally damaging adjacent tissue. The temperature range at which this occurs is from about 50 C to about 80 C. In this range, the conductive nerve tissue in the wall of the LA undergoes cellular necrosis while avoiding the gross tissue trauma, and resultant cellular proliferation that results, from vaporization or charring.

The control algorithm for generator 6005 may detect contact with tissue by sensing impedance levels at electrodes. The algorithm selectively energizes electrodes when the treatment is initiated. Individual control of electrodes may be accomplished by modulating a time and/or level of power in accordance with the control algorithm and feedback sensed at the electrodes and/or temperature sensors. The algorithm may use any of temperature, voltage, current, and impedance, or any combination thereof, as control variables in the algorithm. The application of bipolar RF energy during the course of a treatment ranges from approximately 0.25 W to approximately 25 W of power for a total treatment time from approximately 10 seconds to approximately 2 minutes. During the application of energy, the control algorithm senses whether the control variables are within defined limits according to the software program and feedback. When a variable is outside of its limits, the energy applied to an individual electrode is modulated by increasing, decreasing, or halting applied energy in accordance with the limits of the algorithm equation and during the segment of cycle time for which the modulation condition exists (such as microseconds, milliseconds, seconds). This control method is applied over the course of the treatment period until the treatment endpoint is reached. The treatment endpoint may be any one or more of time, temperature, and impedance. The energy dosage necessary to achieve an efficacious denervation varies by the type of body lumen involved and the energy delivery surface configuration being used. In the case of a PV isolation procedure performed in the antrum region of the LA, the antrum ranges in diameter from about 3 cm to about 10 cm. The LA is heavily perfused with blood and provides an attractive location for isolating lesion formation further assisting to preserve a mild heating that avoids stenosis, phrenic nerve damage, or esophageal damage.

Alternately, energy delivery surfaces 3032 may be configured to be electrodes delivering monopolar RF energy. A common ground may be one of the electrodes, which in turn may optionally be varied by the control algorithm so as to select different electrodes as the ground during cycle time periods over the course of treatment, or an external grounding pad (not shown) may be employed. Conductors (not shown) passing through catheter body 1004 electrically connect the electrodes to a RF generator and controller 6005 via a catheter hub 6000 and an electrical connector 6004. The electrodes are optionally individually configured to be energized and controlled in a modulated fashion to maintain a treatment temperature in accordance with a control algorithm programmed in the software memory of controller 6005.

In an additional monopolar electrode configuration, the struts 3031 may themselves be conductive and areas adjacent to electrode surfaces 3032 on struts 3031 are insulated from conducting energy to tissue of the LA.

The application of monopolar RF energy during the course of a treatment ranges from approximately 0.25 W to approximately 100 W of power for a total treatment time of up to approximately 5 minutes.

In another embodiment, the energy delivery surfaces 3032 on struts 3031 are an array of ultrasound transducers. Ultrasound transducers are optionally individually configured to be energized and controlled in a modulated fashion to maintain a treatment temperature in accordance with a control algorithm. The ultrasound transducers may produce focused or unfocused ultrasound.

Referring now to FIGS. 8,11, and 13, a balloon catheter device 1030 is shown at the distal end of catheter 1004. Balloons may range in expanded diameter from about 3 cm to about 10 cm. A ring-like energy delivery surface 1032 is positioned toward the distal end of balloon 1031. Adjacent or integrated with energy delivery surfaces 1032, one or more optional temperature sensors may be included. Temperature sensors may be thermistors or thermocouples and may be in direct or indirect contact with tissue and/or the energy delivery surface 1032. Conductors (not shown) run proximally through catheter body 1004 and operatively connect the energy delivery surfaces to an energy source and controller. FIG. 8 shows a catheter system 6010 with an integrated energy source and controller 6005. Catheter body 1004 is operatively connected to power source 6005 by a connector 6004 such that conductors pass through a port 6002 of a catheter hub 6000. Catheter hub 6000 may have a guidewire and/or fluid conducting port 6003 in communication with lumens in catheter body 1004. Catheter hub 6000 may have an inflation port 6001 in communication with lumens in catheter body 1004. The configurations of ports in catheter hub 6000 and lumens in catheter body 1004 may depend on the structural embodiment at the distal end of the catheter where the energy surfaces are located. For example, catheter body 1004 would have an inflation lumen for embodiments where a balloon is located at its distal end, while baskets, coils and probes would not require an inflation lumen but may be configured to include a lumen for guidewires. aspiration and/or perfusion. A guidewire lumen would be a preferred embodiment of catheter body 1004 given that over-the-wire and rapid exchange configurations are standard in catheter-based interventional tools.

Energy delivery surface 1032 may be an expandable ring (made of nickel titanium or other flexible materials known in the art), a wire which may optionally be wrapped in a coil to aid in expansion, a flexible circuit, or printed onto balloon 1031 with conductive media such as ink. In embodiments where energy delivery surface 1032 is an expandable ring, the ring may be a plurality of individual segments that may function as individual electrodes.

In some embodiments, the energy delivery surface 1032 is segmented and configured to deliver bipolar RF energy. Conductors (not shown) passing through catheter body 1004 electrically connect the electrodes to a RF generator and controller 6005 via a catheter hub 6000 and an electrical connector 6004. The segments of energy delivery surface 1032 are individually configured to be energized and controlled in a modulated fashion so as to precisely maintain a treatment temperature in accordance with a control algorithm programmed in the software memory of controller 6005. The treatment energy is applied in accordance with the treatment algorithm and a denervating energy treatment is delivered to accomplish isolation of PV by creating a pattern of lesions corresponding to the position of the electrodes. The resultant pattern of lesions 2003 is distributed at point locations about the circumference of the antrum region of the LA, covering substantially the complete circumference of antrum.

The denervating energy treatment is applied in the form of a mild heating of tissue which avoids the deleterious damaging effects of tissue vaporization or tissue charring by delivering energy as a therapeutic dose. A denervating energy treatment is sufficient to cause the denaturing of targeted tissue while applying energy at a level that avoids thermally damaging adjacent tissue. The temperature range at which this occurs is from about 50 C to about 80 C. In this range, the conductive nerve tissue in the wall of the LA undergoes cellular necrosis while avoiding the gross tissue trauma, and resultant cellular proliferation that results, from vaporization or charring.

The control algorithm for generator 6005 may detect contact with tissue by sensing impedance levels at electrodes. The algorithm selectively energizes electrodes when the treatment is initiated. Individual control of electrodes may be accomplished by modulating a time and/or level of power in accordance with the control algorithm and feedback sensed at the electrodes and/or temperature sensors. The algorithm may use any of temperature, voltage, current, and impedance, or any combination thereof, as control variables in the algorithm. The application of bipolar RF energy during the course of a treatment ranges from approximately 0.25 W to approximately 25 W of power for a total treatment time from approximately 10 seconds to approximately 2 minutes. During the application of energy, the control algorithm senses whether the control variables are within defined limits according to the software program and feedback. When a variable is outside of its limits, the energy applied to an individual electrode is modulated by increasing, decreasing, or halting applied energy in accordance with the limits of the algorithm equation and during the segment of cycle time for which the modulation condition exists (such as microseconds, milliseconds, seconds). This control method is applied over the course of the treatment period until the treatment endpoint is reached. The treatment endpoint may be any one or more of time, temperature, and impedance. The energy dosage necessary to achieve an efficacious denervation varies by the type of body lumen involved and the energy delivery surface configuration being used. In the case of a PV isolation procedure performed in the antrum region of the LA, the antrum ranges in diameter from about 3 cm to about 10 cm. The LA is heavily perfused with blood and provides an attractive location for isolating lesion formation further assisting to preserve a mild heating that avoids stenosis, phrenic nerve damage, or esophageal damage.

Alternately, energy delivery surface 3032 may be configured to s deliver monopolar RF energy. A common ground may be an external grounding pad (not shown). Conductors (not shown) passing through catheter body 1004 electrically connect the energy delivery surface 1032 to a RF generator and controller 6005 via a catheter hub 6000 and an electrical connector 6004. Energy delivery surface 1032 may be energized and controlled in a modulated fashion to maintain a treatment temperature in accordance with a control algorithm programmed in the software memory of controller 6005.

The application of monopolar RF energy during the course of a treatment ranges from approximately 0.25 W to approximately 100 W of power for a total treatment time of up to approximately 5 minutes.

Referring again to FIGS. 8 and 13, in another embodiment, energy delivery surface 1032 may be cryogenic delivery surfaces positioned either on the outer surface or the inner surface of balloon 1031. The cryogenic delivery surface 1032 is tubular in nature so as to conduct the cryogen through a fluid transmitting lumen, with a hypotube construction being an example of a cryogenic delivery surface 1032. Optionally, portions of the cryogenic delivery surface 1032 may be insulated to allow for focused delivery of treatment energy at lesion locations in a pattern of point or line locations. Control of cryogenic delivery surface 1032 may be accomplished by modulating a time and/or level of cryogenic delivery in accordance with the generator 6005 control algorithm and feedback sensed at cryogenic delivery surfaces and/or temperature sensors. The algorithm may use any of temperature, impedance, cryogen flow rate, cryogen flow time, or any combination thereof, as control variables in the algorithm. The application of energy during the course of a treatment is based on the thermal properties of the specific cryogen being used, any of the now known cryogens for use in AF therapies—as well as any as yet untried cryogens—being suitable, for a total treatment time from approximately 10 seconds or more. In cryogenic embodiments of the present invention, tissue treatment temperatures are below 0 C (as opposed to approximately 50 C to approximately 80 C in non-cryogenic embodiments). During cryogenic delivery, the control algorithm senses whether the control variables are within defined limits according to the software program and feedback. When a variable is outside of its limits, the cryogenic delivery applied to energy delivery surfaces is modulated by increasing, decreasing, or halting applied cryogenic delivery in accordance with the limits of the algorithm equation and during the segment of cycle time for which the modulation condition exists (such as microseconds, milliseconds, seconds). This control method is applied over the course of the treatment period until the treatment endpoint is reached. The treatment endpoint may be any one or more of time, temperature, and impedance.

Referring now to FIGS. 8,11, and 14, in another embodiment of the present invention, the catheter-based system 6010 is configured with a coil-like expandable structure 4020 at the distal end of catheter body 1004 ranging in expanded diameter from about 3 cm to about 10 cm, which includes energy delivery surfaces 4022 at points along the body 4021 of the coil, and which are positioned to create a series of energy delivery locations that create a plurality of individual lesions 2003 that are continuous or substantially continuous about the inner circumference of the antrum region of the LA. Adjacent or integrated with energy surfaces 4022, one or more optional temperature sensors may be included. Temperature sensors may be thermistors or thermocouples and may be in direct or indirect contact with tissue and/or the energy delivery surfaces 4022.

Referring now to FIGS. 7 and 8, in another embodiment, the energy delivery surface 5002 on probe body 5001 is a cryogenic delivery system controlled in a modulated fashion to maintain a treatment temperature in accordance with a control algorithm. Control of cryogenic delivery surface 5002 may be accomplished by modulating time and/or level of cryogenic delivery in accordance with the generator 6005 control algorithm and feedback sensed at cryogenic delivery surfaces and/or temperature sensors. The algorithm may use any of temperature, impedance, cryogen flow rate, cryogen flow time, or any combination thereof, as control variables in the algorithm. The application of energy during the course of a treatment is based on the thermal properties of the specific cryogen being used, any of the now known cryogens for use in AF therapies—as well as any as yet untried cryogens—being suitable, for a total treatment time from approximately 1 second or more. In cryogenic embodiments of the present invention, tissue treatment temperatures are below 0 C (as opposed to approximately 50 C to approximately 80 C in non-cryogenic embodiments). During cryogenic delivery, the control algorithm senses whether the control variables are within defined limits according to the software program and feedback. When a variable is outside of its limits, the cryogenic energy applied to energy delivery surfaces is modulated by increasing, decreasing, or halting applied cryogenic delivery in accordance with the limits of the algorithm equation and during the segment of cycle time for which the modulation condition exists (such as microseconds, milliseconds, seconds). This control method is applied over the course of the treatment period until the treatment endpoint is reached. The treatment endpoint may be any one or more of time, temperature, and impedance.

In some embodiments, the energy delivery surfaces 4022 are electrodes configured to deliver bipolar RF energy. Conductors (not shown) passing through catheter body 1004 electrically connect the electrodes to a RF generator and controller 6005 via a catheter hub 6000 and an electrical connector 6004. The electrodes are individually configured to be energized and controlled in a modulated fashion so as to precisely maintain a treatment temperature in accordance with a control algorithm programmed in the software memory of controller 6005. The treatment energy is applied in accordance with the treatment algorithm and a denervating energy treatment is delivered to accomplish isolation of PV by creating a pattern of lesions 2003 corresponding to the position of the electrodes. The resultant pattern of lesions is distributed at point locations about the circumference and length of PV, and when viewed in a plane perpendicular to the length of PV cover substantially the complete circumference of PV.

The control algorithm for generator 6005 may detect contact with tissue by sensing impedance levels at electrodes. The algorithm selectively energizes electrodes when the treatment is initiated. Individual control of electrodes may be accomplished by modulating a time and/or level of powering in accordance with the control algorithm and feedback sensed at the electrodes and/or temperature sensors. The algorithm may use any of temperature, voltage, current, and impedance as control variables in the algorithm. The application of bipolar RF energy during the course of a treatment ranges from approximately 0.25 W to approximately 25 W of power for a total treatment time from approximately 10 seconds to approximately 2 minutes. During the application of energy, the control algorithm senses whether the control variables are within defined limits according to the software program and feedback. When a variable is outside of its limits, the energy applied to an individual electrode is modulated by increasing, decreasing, or halting applied energy in accordance with the limits of the algorithm equation and during the segment of cycle time for which the modulation condition exists (such as microseconds, milliseconds, seconds). This control method is applied over the course of the treatment period until the treatment endpoint is reached. The treatment endpoint may be any one or more of time, temperature, and impedance. The energy dosage necessary to achieve an efficacious denervation varies by the type of body lumen involved and the energy delivery surface configuration being used. In the case of a PV isolation procedure, the PV ranges in diameter from about 5 mm to about 16 mm and the PV is heavily perfused with blood. As compared to delivery of energy in a peripheral vessel or delivery of energy in a renal artery, energy delivery surfaces may be larger in size and/or higher in number in order to provide the necessary lesion pattern while seeking to preserve a mild heating that avoids stenosis, phrenic nerve damage, or esophageal damage.

Alternately, energy delivery surfaces 4022 may be electrodes configured to deliver monopolar RF energy. A common ground may be one of the electrodes, which in turn may optionally be varied by the control algorithm so as to select different electrodes as the ground during cycle time periods over the course of treatment, or an external grounding pad (not shown) may be employed. Conductors (not shown) passing through catheter body 1004 electrically connect the electrodes to an RF generator and controller 6005 via a catheter hub 6000 and an electrical connector 6004. The electrodes are optionally individually configured to be energized and controlled in a modulated fashion to maintain a treatment temperature in accordance with a control algorithm programmed in the software memory of controller 6005.

In an additional monopolar electrode configuration, the coil body 4021 may itself be conductive and the spaces between electrode surfaces on the coil body are insulated from conducting energy to tissue of the PV.

The application of monopolar RF energy during the course of a treatment ranges from approximately 0.25 W to approximately 100 W of power for a total treatment time of up to approximately 5 minutes.

In another embodiment, the energy delivery surfaces 4022 on the coil body 4021 are an array of ultrasound transducers. Ultrasound transducers 4022 are optionally individually configured to be energized and controlled in a modulated fashion to maintain a treatment temperature in accordance with a control algorithm. The ultrasound transducers 4022 may produce focused or unfocused ultrasound.

From the foregoing, it will be appreciated that, although specific embodiments of the invention have been described herein for the purpose of illustration, various modifications may be made without deviating from the spirit and scope of the invention. Accordingly, the present invention is not limited except as by the appended claims.

All patents, patent applications, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Additionally, all claims in this application, and all priority applications, including but not limited to original claims, are hereby incorporated in their entirety into, and form a part of, the written description of the invention. Applicant reserves the right to physically incorporate into this specification any and all materials and information from any such patents, applications, publications, scientific articles, web sites, electronically available information, and other referenced materials or documents. Applicant reserves the right to physically incorporate into any part of this document, including any part of the written description, the claims referred to above including but not limited to any original claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features reported and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims. Other embodiments are within the following claims.

1. A method for isolating a pulmonary vein for the treatment of atrial fibrillation, the method comprising:
(a) accessing the pulmonary vein with a distal portion of a catheter-based device by using an interventional technique;
(b) deploying a structure at the distal end of the catheter, comprised to include a plurality of energy delivery surfaces, such that at least one energy delivery surface is in contact with the tissue of the pulmonary vein;
(c) applying a denervating energy treatment to the tissue of the wall of the pulmonary vein adjacent the energy delivery surfaces in contact with the pulmonary vein;
(d) modulating the denervating energy treatment so as to avoid charring or vaporizing of tissue by maintaining a temperature from approximately 50 C to approximately 80 C adjacent an energy delivery surface during the period which energy is provided to the energy delivery surface;
(e) forming a plurality of discontinuous lesions about the ostial portion of the pulmonary vein having both a circumferential and axial offset between immediately adjacent individual lesions, wherein individual lesions are positioned to be approximately continuous about the circumference of the pulmonary vein when viewed from a plane perpendicular to the length of the pulmonary vein and positioned to be circumferentially and axially offset from one another when viewed along the length of the pulmonary vein, and wherein the pattern of lesions isolates the pulmonary vein in an atrial fibrillation treatment.

2. The method of claim 1, wherein the catheter-based device is operatively coupled to a system further comprised of an integrated generator and controller, wherein the generator and controller modulates the energy delivery surfaces using a software-based algorithm.

3. The method of claim 1, wherein sensed feedback is used to provide input for a denervating energy delivery surface modulation calculation.

4. The method of claim 3, wherein the sensed feedback includes one or more of temperature, voltage, current, impedance.

5. The method of claim 1, wherein treatment time ranges from about 10 seconds to about 5 minutes.

6. The method of claim 1, wherein treatment power ranges from about 0.25 Watts to about 100 Watts.

7. The method of claim 1, wherein the structure at the distal end of the catheter is an inflatable and collapsible balloon comprised to include one or more energy delivery surfaces thereon, the individual energy delivery surfaces being circumferentially and axially offset from the immediately adjacent individual energy delivery surfaces.

8. The method of claim 7, wherein the energy delivery surfaces are radiofrequency electrodes having a flexible circuit construction.

9. The method of claim 8, wherein the electrodes deliver bipolar radiofrequency energy.

10. The method of claim 8, wherein the electrodes deliver monopolar radiofrequency energy.

11. The method of claim 7, wherein the energy delivery surfaces are ultrasound transducers.

12. The method of claim 11, wherein the ultrasound transducers deliver focused ultrasound energy.

13. The method of claim 11, wherein the ultrasound transducers deliver unfocused ultrasound energy.

14. The method of claim 7, wherein the balloon diameter is between about 5 mm and about 16 mm.

15. The method of claim 14, wherein the balloon diameter tapers from its proximal end to its distal end.

16. The method of claim 7, wherein the balloon is further comprised to include one or more temperature sensors.

17. The method of claim 1, wherein the structure at the distal end of the catheter is an expandable and collapsible basket, having a proximal basket diameter which is larger than a distal basket diameter, being further comprised to include one or more energy delivery surfaces thereon, the individual energy delivery surfaces being circumferentially and axially offset from the immediately adjacent individual energy delivery surfaces.

18. The method of claim 17, wherein the distal end of the basket is open-ended.

19. The method of claim 17, wherein the distal end of the basket is closed-ended.

20. The method of claim 17, wherein the energy delivery surfaces are flexible radiofrequency electrodes.

21. The method of claim 20, wherein the electrodes deliver bipolar radiofrequency energy.

22. The method of claim 20, wherein the electrodes deliver monopolar radiofrequency energy.

23. The method of claim 17, wherein the energy delivery surfaces are ultrasound transducers.

24. The method of claim 23, wherein the ultrasound transducers deliver focused ultrasound energy.

25. The method of claim 23, wherein the ultrasound transducers deliver unfocused ultrasound energy.

26. The method of claim 17, wherein the basket diameter is between about 5 mm and about 16 mm.

27. The method of claim 17, wherein the basket construction is comprised of nickel-titanium.

28. The method of claim 17, wherein the basket is further comprised to include one or more temperature sensors.

29. The method of claim 1, wherein the structure at the distal end of the catheter is an expandable and collapsible coil having a proximal coil diameter which is larger than the distal coil diameter, being further comprised to include one or more energy delivery surfaces thereon, the individual energy delivery surfaces being circumferentially and axially offset from the immediately adjacent individual energy delivery surfaces.

30. The method of claim 29, wherein the energy delivery surfaces are radiofrequency electrodes.

31. The method of claim 30, wherein the electrodes deliver bipolar radiofrequency energy.

32. The method of claim 30, wherein the electrodes deliver monopolar radiofrequency energy.

33. The method of claim 29, wherein the energy delivery surfaces are ultrasound transducers.

34. The method of claim 33, wherein the ultrasound transducers deliver focused ultrasound energy.

35. The method of claim 33, wherein the ultrasound transducers deliver unfocused ultrasound energy.

36. The method of claim 29, wherein the coil diameter is between about 5 mm and about 16 mm.

37. The method of claim 29, wherein the coil is further comprised to include one or more temperature sensors.

38. The method of claim 1, wherein the structure at the distal end of the catheter is a probe comprised to include one or more energy delivery surfaces thereon.

39. The method of claim 38, wherein the energy delivery surfaces are radiofrequency electrodes.

40. The method of claim 39, wherein the electrodes deliver bipolar radiofrequency energy.

41. The method of claim 39, wherein the electrodes deliver monopolar radiofrequency energy.

42. The method of claim 38, wherein the energy delivery surfaces are ultrasound transducers.

43. The method of claim 42, wherein the ultrasound transducers deliver focused ultrasound energy.

44. The method of claim 42, wherein the ultrasound transducers deliver unfocused ultrasound energy.

45. The method of claim 38, wherein the probe is configured to be deflectable to any angle up to approximately 90 degrees from the undeflected position.

46. The method of claim 38, wherein the probe is further comprised to include one or more temperature sensors.

47. A method for isolating a pulmonary vein for the treatment of atrial fibrillation, the method comprising:
(a) accessing the pulmonary vein with a distal portion of a catheter-based device by using an interventional technique;
(b) deploying a structure at the distal end of the catheter, comprised to include a plurality of cryogenic delivery surfaces, such that at least one cryogenic delivery surface is in contact with the tissue of the pulmonary vein;
(c) applying a cryogenic denervating treatment to the tissue of the wall of the pulmonary vein adjacent the cryogenic delivery surfaces in contact with the pulmonary vein;
(d) modulating the cryogenic denervating treatment so as to avoid damaging tissue adjacent cryogenic delivery surfaces by maintaining a precise treatment temperature adjacent a delivery surface during the period which a cryogen is provided to the cryogenic delivery surface;
(e) forming a plurality of discontinuous lesions about the ostial portion of the pulmonary vein having both a circumferential and axial offset between immediately adjacent individual lesions, wherein individual lesions are positioned to be approximately continuous about the circumference of the pulmonary vein when viewed from a plane perpendicular to the length of the pulmonary vein and positioned to be circumferentially and axially offset from one another when viewed along the length of the pulmonary vein, and wherein the pattern of lesions isolates the pulmonary vein in an atrial fibrillation treatment.

48. The method of claim 47, wherein the catheter-based device is operatively coupled to a system further comprised of an integrated generator and controller, wherein the generator and controller modulate the cryogenic delivery using a software-based algorithm.

49. The method of claim 48, wherein sensed feedback is used to provide input for a denervating cryogenic delivery surface modulation calculation.

50. The method of claim 49, wherein the sensed feedback includes one or more of temperature, time, voltage, current, impedance.

51. The method of claim 47, wherein the structure at the distal end of the catheter is an inflatable and collapsible balloon having a proximal balloon diameter which is larger than a distal balloon diameter, being further comprised to include one or more cryogenic delivery surfaces thereon, the individual cryogenic delivery surfaces being circumferentially and axially offset from the immediately adjacent individual cryogenic delivery surfaces.

52. The method of claim 51, wherein the cryogenic delivery surface is comprised of a hypotube.

53. The method of claim 52, wherein portions of the surface of the hypotube are insulated so as to focus cryogenic treatment at the lesion locations.

54. A method for isolating a pulmonary vein for the treatment of atrial fibrillation, the method comprising:
(a) accessing the antrum region of the left atrium, in proximity to an inferior pulmonary vein and a superior pulmonary vein, with a distal portion of a catheter-based device by using an interventional technique;
(b) deploying a structure at the distal end of the catheter, comprised to include a plurality of energy delivery surfaces, such that at least one energy delivery surface is in contact with the tissue of the antrum regions of the left atrium;
(c) applying a denervating energy treatment to the tissue of the wall of the antrum region of the left atrium adjacent the energy delivery surfaces in contact with the antrum region;
(d) modulating the denervating energy treatment so as to avoid charring or vaporizing of tissue by maintaining a temperature from approximately 50 C to approximately 80 C adjacent an energy delivery surface during the period which energy is provided to the energy delivery surface;
(e) forming a plurality of lesions about the antrum region, wherein individual lesions are positioned to be approximately continuous about the circumference of the antrum region, and wherein the pattern of lesions isolates the pulmonary veins in an atrial fibrillation treatment.

55. The method of claim 54, wherein the catheter-based device is operatively coupled to a system further comprised of an integrated generator and controller, wherein the generator and controller modulate the energy delivery surfaces using a software-based algorithm.

56. The method of claim 54, wherein sensed feedback is used to provide input for a denervating energy delivery surface modulation calculation.

57. The method of claim 56, wherein the sensed feedback includes one or more of temperature, voltage, current, impedance.

58. The method of claim 54, wherein treatment time ranges from about 10 seconds to about 5 minutes.

59. The method of claim 54, wherein treatment power ranges from about 0.25 Watts to about 100 Watts.

60. The method of claim 54, wherein the structure at the distal end of the catheter is an inflatable and collapsible balloon comprised to include one or more energy delivery surfaces thereon.

61. The method of claim 60, wherein the energy delivery surfaces are radiofrequency electrodes having a flexible circuit construction.

62. The method of claim 61, wherein the electrodes deliver bipolar radiofrequency energy.

63. The method of claim 61, wherein the electrodes deliver monopolar radiofrequency energy.

64. The method of claim 60, wherein the energy delivery surfaces are ultrasound transducers.

65. The method of claim 64, wherein the ultrasound transducers deliver focused ultrasound energy.

66. The method of claim 64, wherein the ultrasound transducers deliver unfocused ultrasound energy.

67. The method of claim 60, wherein the balloon diameter is between about 3 cm and about 10 cm.

68. The method of claim 60, wherein the balloon is further comprised to include one or more temperature sensors.

69. The method of claim 54, wherein the structure at the distal end of the catheter is an expandable and collapsible basket, being further comprised to include one or more energy delivery surfaces thereon.

70. The method of claim 69, wherein the distal end of the basket is open-ended.

71. The method of claim 69, wherein the distal end of the basket is closed-ended.

72. The method of claim 69, wherein the energy delivery surfaces are flexible radiofrequency electrodes.

73. The method of claim 72, wherein the electrodes deliver bipolar radiofrequency energy.

74. The method of claim 72, wherein the electrodes deliver monopolar radiofrequency energy.

75. The method of claim 69, wherein the energy delivery surfaces are ultrasound transducers.

76. The method of claim 75, wherein the ultrasound transducers deliver focused ultrasound energy.

77. The method of claim 75, wherein the ultrasound transducers deliver unfocused ultrasound energy.

78. The method of claim 69, wherein the basket diameter is between about 3 cm and about 10 cm.

79. The method of claim 69, wherein the basket construction is comprised of nickel-titanium.

80. The method of claim 69, wherein the basket is further comprised to include one or more temperature sensors.

81. The method of claim 54, wherein the structure at the distal end of the catheter is an expandable and collapsible coil, being further comprised to include one or more energy delivery surfaces thereon.

82. The method of claim 81, wherein the energy delivery surfaces are radiofrequency electrodes.

83. The method of claim 82, wherein the electrodes deliver bipolar radiofrequency energy.

84. The method of claim 82, wherein the electrodes deliver monopolar radiofrequency energy.

85. The method of claim 81, wherein the energy delivery surfaces are ultrasound transducers.

86. The method of claim 85, wherein the ultrasound transducers deliver focused ultrasound energy.

87. The method of claim 85, wherein the ultrasound transducers deliver unfocused ultrasound energy.

88. The method of claim 81, wherein the coil diameter is between about 3 cm and about 10 cm.

89. The method of claim 81, wherein the coil is further comprised to include one or more temperature sensors.

90. A method for isolating a pulmonary vein for the treatment of atrial fibrillation, the method comprising:
(a) accessing the antrum region of the left atrium, in proximity to an inferior pulmonary vein and a superior pulmonary vein, with a distal portion of a catheter-based device by using an interventional technique;
(b) deploying a structure at the distal end of the catheter, comprised to include a plurality of cryogenic delivery surfaces, such that at least one cryogenic delivery surface is in contact with the tissue of the pulmonary vein;
(c) applying a cryogenic denervating treatment to the tissue of the wall of the antrum region of the left atrium adjacent the cryogenic delivery surfaces in contact with the antrum region;
(d) modulating the cryogenic denervating treatment so as to avoid damaging tissue adjacent cryogenic delivery surfaces by maintaining a precise treatment temperature adjacent a delivery surface during the period which a cryogen is provided to the cryogenic delivery surface;
(e) forming a plurality of lesions about the antrum region, wherein individual lesions are positioned to be approximately continuous about the circumference of the antrum region, and wherein the pattern of lesions isolates the pulmonary veins in an atrial fibrillation treatment.

91. The method of claim 90, wherein the catheter-based device is operatively coupled to a system further comprised of an integrated generator and controller, wherein the generator and controller modulate the cryogenic delivery using a software-based algorithm.

92. The method of claim 91, wherein sensed feedback is used to provide input for a denervating cryogenic delivery surface modulation calculation.

93. The method of claim 92, wherein the sensed feedback includes one or more of temperature, time, voltage, current, impedance.

94. The method of claim 90, wherein the structure at the distal end of the catheter is an inflatable and collapsible balloon, being further comprised to include one or more cryogenic delivery surfaces thereon.

95. The method of claim 94, wherein the cryogenic delivery surface is comprised of a hypotube.

96. The method of claim 95, wherein portions of the surface of the hypotube are insulated so as to focus cryogenic treatment at the lesion locations.

97. The method of claim 47, wherein the device at the distal end of the catheter is a probe comprised to include one or more cryogenic delivery surfaces thereon.

98. The method of claim 90, wherein the device at the distal end of the catheter is a probe comprised to include one or more cryogenic delivery surfaces thereon.
